(19) United States
(12) Patent Application Publication
(10) Pub. No.: US 2025/0222451 A1
(43) Pub. Date: Jul. 10, 2025

(54) ELECTRONIC ASSEMBLY OF LONG DNA MOLECULES

(71) Applicant: Avery Digital Data, Inc., San Diego, CA (US)

(72) Inventors: Barry Merriman, San Diego, CA (US); Ryan de Ridder, San Diego, CA (US); Matthew Holden, San Diego, CA (US); Sang Heon Lee, San Diego, CA (US); Kyle Gustafson, Glendale, CA (US)

(21) Appl. No.: 18/846,594

(22) PCT Filed: Dec. 19, 2022
(86) PCT No.: PCT/US2022/053406

§ 371 Date: Sep. 12, 2024

Related U.S. Application Data
(60) Provisional application No. 62/291,124, filed on Feb. 4, 2016.

(51) Int. Cl. B01L 3/00; C12N 15/10

(52) U.S. Cl. CPC B01L 3/502761; C12N 15/1065; B01L 2200/0663; B01L 2300/0645; B01L 2300/0896; B01L 2400/0415

(57) ABSTRACT

A DNA assembly device is disclosed. The device contains a chip having at least one nanochannel integrated therein, the at least one nanochannel having a reaction zone where ends of at least two distinct DNA molecules can be maintained proximate to one another; and circuits and electrodes for controlling movement of the at least two distinct DNA molecules. Methods of use are and systems using multiple nanochannels are also disclosed.

### CROSS-REFERENCE TO RELATED APPLICATIONS

This application claims priority to and the benefit of U.S. Provisional Patent Application Ser. No. 63/291,124, filed Dec. 17, 2021, and entitled “Electronic Assembly of Long DNA Molecules for Synthetic Biology”, the disclosure of which is incorporated herein by reference in its entirety.

### FIELD

This disclosure is in the field of synthetic biology. In particular, this disclosure describes nanochannel devices for the assembly of long DNA molecules, the use of semiconductor chips for such devices, and methods for using the same in synthetic biology.

### BACKGROUND

The field of synthetic biology is broadly concerned with engineering biological processes to achieve desired functional endpoints. One major area of focus is in modifying the DNA of organisms as a means of engineering their functional properties. In particular, this includes the need to synthesize the DNA sequences that correspond to individual genes, sets of genes, and larger genomic DNA constructs such as plasmid or chromosomal elements, up to the extremes of entire genomes of artificial organisms.

The standard commercial method of primary DNA chemical synthesis, the phosphoramidite method, is in practice limited to making DNA oligomers, up to several hundred bases in length. In contrast, common coding gene lengths are in the range of 1000-3000 bases, and thus even for producing such genes, primary synthesis oligomers must be further joined to make these longer fragments, at the scale of joining ˜10-˜100 such oligomers. For the exonic format of genes, such as in animals, the genomic extent of a gene is often on the order of 10,000-100,000 bases, and thus fabricating genes in this format would require joining DNA on an order of magnitude larger scale. Similarly, the set of genes that comprise a biochemical synthesis pathway may typically be 10-100 genes, and packaging these into a single DNA segment “cassette” would require joining together fragments to achieve lengths of ˜10,000-˜100,000 bases. Making the genomes of the smallest single celled organisms, such as mycoplasma, also requires making ˜100,000 base fragments, and the larger chromosomes of bacteria, fungi, or other simple organisms would require making DNA in the 1 megabase-10 megabase regime. Thus, the field of synthetic biology requires methods to produce ultimate products in the length range of 10,000-10,000,000 bases.

In practice, the classical method of Gibson assembly, and related methods, are used to assemble oligomers towards these greater lengths. Such methods generally take the input fragments, and pool them in an assembly reaction in solution. Such approaches are limited in the length and sequence content of the resulting products. The result is that using such methods, there is substantial process complexity, high cost, long processing time, along with substantial sequence content restrictions, and a high rate of failed assembly reactions. Therefore, new methods of assembly not having these deficiencies are beneficial for the future of bioengineering.

Nanochannels are nano-fluidic structures that are used to isolate small numbers of molecules. Such constructs were introduced in the 1990's. They consist of a long fluid channel, where the width and depth are on the nanometer (nm) scale, in the range of 10-200 nm, and the channel length dimension is much larger, typically in the range of microns to millimeters. Such channels, when loaded with solution, and covered, can also be loaded with molecules, such as through the application of pressure (to drive solution in) and/or voltages, to drive charged molecules into the channels. Studying the behavior of molecules loaded into nanochannels is the basic aspect of the field of nanofluidics.

### SUMMARY

In an aspect, a DNA assembly device is disclosed. The device includes a chip having at least one nanochannel integrated therein, the at least one nanochannel having a reaction zone where ends of at least two distinct DNA molecules can be maintained proximate to one another; and circuits and electrodes for controlling movement of the at least two distinct DNA molecules.

In embodiments, movement of the at least two DNA molecules is controlled electrically. In embodiments, the DNA molecules have lengths of 50-1,000 bases, or 1,000-10,000 bases, or 10,000-100,000 bases, or 100,000-1,000,000 bases, or 1,000,000-10,000,000 bases, or more than 10,000,000 bases. In certain embodiments, the chip contains multiple nanochannels, each of which can operate independently. In certain embodiments, the chip is a CMOS chip. In certain embodiments, the top of the at least one nanochannel is sealed.

In embodiments, the surfaces of the at least one nanochannel are functionalized. In embodiments, at least one portion of the at least one nanochannel is constricted. In certain embodiments, the reaction zone is fluidically connected to an end of the at least one nanochannel. In certain embodiments, the reaction zone is configured along the length of the at least one nanochannel.

In another aspect, a system is disclosed that includes first and second devices as disclosed herein, wherein each of the respective nanochannels are fluidically connected.

In another aspect, a system is disclosed that includes a device as disclosed herein having multiple nanochannels. In this system, the device contains a network of nanochannels. In embodiments, the number of networked nanochannels is 1-10, or 10-100, or 100-1000, or 1000-10,000, or 10,000-100,000, or 100,000-1,000,000, or more than 1,000,000. In embodiments, the chip is a CMOS chip.

In another aspect, a method of joining two DNA molecules is disclosed. The method involves introducing a first DNA molecule to a first nanochannel, wherein the first nanochannel contains a reaction zone; introducing a second DNA molecule strand to a second nanochannel, wherein the second nanochannel is also connected to the reaction zone; and electrically controlling the movement of the first and second DNA molecules strands to the reaction zone where the first and second nucleotide strands undergo a series of joining reactions. In certain embodiments, the first and second nanochannels are the same. In certain embodiments, each of the DNA molecules contain a mechanical blocking group. In certain embodiments, the method further involves removing the mechanical blocking groups following the joining of the first and second DNA molecules. In certain embodiments, the DNA molecules contain unique barcode elements for tracking each of the DNA molecules. The DNA molecules may have lengths of 50-1,000 bases, or 1,000-10,000 bases, or 10,000-100,000 bases, or 100,000-1,000,000 bases, or 1,000,000-10,000,000 bases, or more than 10,000,000 bases.

Further, and without limiting the foregoing, disclosed herein are nanochannel device compositions that are useful for joining single molecules of DNA to form longer contiguous single DNA molecules. This has the advantage that nanochannels can hold and move long single molecules of DNA with much less risk of the DNA strand breaking, as happens commonly when fragments of DNA are manipulated by classical bulk solution phase methods such as pipetting, mixing, centrifuging, and gel electrophoresis.

Further, disclosed herein are nanochannel devices and methods that can be used to join given single molecule strands of DNA to form longer continuous single molecule DNA strands. This includes devices and methods for joining strands that may be in the length range of 100-1000 bases, or in the range of 1,000-10,000 bases, or 10,000-100,000 bases, or 100,000-1,000,000 bases, or 1,000,000-10,000,000 bases, or strands longer than 10,000,000 bases. This has the advantage that such devices and methods can support the joining of such long strands with much less risk of breaking them or the resulting strand than classical bulk solution methods. This also has the advantage of requiring much less DNA material than classical bulk solution methods, since the operations are done at the single-molecule level. This also has the advantage that such constructs can be tracked and collected at the single molecule level, thereby avoiding the need for subsequent purification or isolation processes.

Further, disclosed herein are nanochannel devices and methods that can be used to join strands through a series of pair-wise joining reactions, that can be used join from 2-10 strands in series, 10-100 strands in series, or 100-1000 strands in series, or more than 1000 strands in series. These pairwise assemblies can be further joined in a hierarchical assembly process. This has the advantage of producing long products while eliminating constraints on the DNA sequence content of the resulting strand, because each segment is added without restriction on how its sequence is related to that of the growing strand, as it is prevented by its extended form in the nanochannel from having physical interactions with the rest of the strand. In contrast, classical assembly methods, such as the well-known bulk phase Gibson assembly, allow the segments and growing strand to interact, which can result in incorrect joins, or interference in such joins. These classical unwanted interactions typically result in constraints whereby joining can only be efficiently performed if the sequences are not too repetitive, low-complexity, or self-complementary.

Further, disclosed herein are nanochannel devices and methods that can be used to join together a series of DNA strands, where each strand in the series is further selectable from a selection of N strand options provided for each segment, all under nanochannel control. Here N may be in the range of 2-10 options, 10-100 options, or 100 or more options, and the strand options may be different for each segment in the series. By these means, the resulting DNA fragment F can the combinatorial structure of F=S1−S2− . . . −Si−. . . −Sn; where each DNA segment Si is selected from a set of Ni≥1 total options Oi={Si,1, Si,2, . . . , Si, Ni}, for i=1, . . . , n. The total number of possible combinations of parts is C=N1×N2× . . . ×Nn, which can be a very large number of possible combinations. The advantage of this approach is that it allows greater efficiency, speed, and low reagent consumption, as well as allowing for much longer products without breakage, and allowing for better tracking of the resulting products, when performing such combinatorial assembly methods from input sets “parts”, as compared to classic bulk fluidic methods. The advantage is also that the nanochannel devices can be embodied in a highly scalable fashion, to enable production of a much larger number of such combinations, and at a greater rate than is possible with classical bulk assembly methods.

Further, disclosed herein are nanochannel devices and methods that can perform the above joining processes in parallel, and under electronic control, such that many such joined strands may be formed in parallel, and rapidly and efficiently through electronic control. This has the advantage of providing scalable devices, that can carry out many such reactions in parallel. The number of parallel strand assembles on one device may be up to 10, up to 100, up to 1000, up to 10,000, or up to 1 million or more.

Further, disclosed herein are complementary metal-oxide semiconductor (CMOS) chips devices that comprise the nanochannels for the above, and which can apply the electronic control of the operations for the nanochannels for the above. This has the advantage that the CMOS chips enjoy of the greatest existing manufacturing base among all types of semiconductor chips, and the greatest capacity for production and low-cost mass manufacturing, providing for both the fundamental cost reductions for deploying the nanochannel DNA assembly processes disclosed, as well as scalability of the number and complexity of such devices that can be implemented on a chip, or per square millimeter of chip area.

Further, disclosed herein are methods of monitoring and checking such nanochannel assembly processes, through various sensor modalities built into the devices. This has the advantage that it can be known whether the steps of assembly, or final assembly, of strands has been properly performed, thereby allowing a higher yield of properly formed target strands.

Further, disclosed herein are systems that make use of such nanochannel devices and methods for the production of many long strands of DNA formed by joining shorter strands. This provides the advantage of enabling end-to-end automated processing, to go from input fragments to output long DNA joined strands, and with tracking of the final output strands.

Further, and without limiting any of the foregoing, disclosed herein are compositions, devices, methods, and systems for the fabrication of long DNA molecules for applications in synthetic biology.

### BRIEF DESCRIPTION OF THE DRAWING FIGURES

FIG. 1 shows a basic nanochannel geometry and critical dimensions.

FIG. 2 shows multiple views of the basic nanochannel geometry.

FIG. 3 shows various top-layer enclosures for nanochannels.

FIG. 4 shows nanochannels arranged in parallel.

FIG. 5 shows large scale arrays of nanochannels arranged in parallel.

FIG. 6 shows nanochannel inlet geometries.

FIG. 7 shows views of pillar arrays for linearizing DNA molecules entering a nanochannel inlet.

FIG. 8 shows elements of nanochannel geometry with changes in direction.

FIG. 9 shows elements of nanochannel geometry with branching of channels.

FIG. 10 shows elements of nanochannel geometry with reaction zones.

FIG. 11 shows the application of voltage used to drive a DNA molecule through the channel.

FIG. 12 shows a top view of a nanochannel inlet intaking DNA molecules over time.

FIG. 13 shows a top view of an array of nanochannels with voltages driving DNA molecules through the channels.

FIG. 14 shows nanochannel junctions and voltages used to move the ends of two DNA molecules into a reaction zone.

FIG. 15 shows two fragments paused in the reaction zone, by removal of the driving voltage.

FIG. 16 shows the joining of two DNA molecules in a nanochannel device.

FIG. 17 shows the use of motion stops within the channels to control the location of the ends of DNA molecules and facilitate joining of ends.

FIG. 18 shows the subsequent controlled transfer of a DNA segment after joining of ends with the use of motion stops.

FIG. 19 shows the use of parallel channels with motion stops to join the ends of multiple fragment pairs in parallel.

FIG. 20 shows the use of multiple motion stops to bring in fragments serially for joining.

FIG. 21 shows the organization of parallel motion stop channels and reaction zones.

FIG. 22 shows the process of loading multiple motion stops with a first DNA end in the reaction zones following FIG. 21.

FIG. 23 shows the process of loading multiple motion stops with a second DNA end in the reaction zones following FIG. 22.

FIG. 24 shows the process of joining multiple DNA end pairs in the reaction zones following FIG. 23.

FIG. 25 shows the output of the multiple joined DNA fragments from the reaction zones following FIG. 24.

FIG. 26 shows a configuration for the serial joining of DNA fragments in nanochannels with motion stops and reaction zones, and the first step of this process.

FIG. 27 shows the second step for the process of serial joining of DNA fragments, following FIG. 26.

FIG. 28 shows the third step for the process of serial joining of DNA fragments, following FIG. 27.

FIG. 29 shows the final output transfer step for the process of serial joining of DNA fragments, following FIG. 28.

FIG. 30 shows elements of nanochannel geometry and process for basic elements of motion control: controlled stop, linear translation, and turning around for a linear DNA fragment.

FIG. 31 shows the process of turning around a linear DNA fragment, using the triple junction element.

FIG. 32 shows a 3-D view of the input port for a nanochannel device.

FIG. 33 shows top and side views of the input port for a nanochannel device.

FIG. 34 shows a side view of the process of inputting DNA fragments into a nanochannel input port.

FIG. 35 shows a top view the process of inputting DNA fragments into a nanochannel input port, happening in parallel in adjacent parallel channels.

FIG. 36 shows a top view the process of inputting DNA fragments into a nanochannel input port, happening in parallel in an arbitrary number of parallel channels, with common driving voltages.

FIG. 37 shows a top view the process of inputting DNA fragments into a nanochannel input port, happening in parallel and with parallel independent control of the driving voltages.

FIG. 38 shows a circuit-schematic view of a nanochannel device for joining one of N optional fragments to an input strand and outputting the final strand.

FIG. 39 shows a circuit-schematic view of a nanochannel device for joining a series of K segments, where each is selected from one of N optional fragments, to an input strand and outputting the final strand.

FIG. 40 shows a circuit-schematic view of a nanochannel device for joining one of N optional fragments to each of S different input strands and outputting the S final strands.

FIG. 41 shows a circuit-schematic view of a nanochannel device for joining a series of K segments, where each is selected from one of N optional fragments, to each of S input strands and outputting the S final strand.

FIG. 42 shows a device that is an R row×C column array of devices from FIG. 41, capable of performing those operations with further R×C-fold parallelism.

FIG. 43 shows a schema for the outermost inputs and outputs to drive the device of FIG. 42, such that all input optional fragments, output strands, and input strands for the entire array are provided from, and output to, the external inlet/outlets reservoirs.

FIG. 44 shows a preferred embodiment for implementation of a nanochannel device on a CMOS chip.

FIG. 45 indicates various types of DNA joining procedures that may be used to join DNA fragments in the reaction zones.

FIG. 46 shows various nanochannel joining strategies for increasing the length of the fragment with N serial joining steps, showing linear and exponential growth of length with joining cycle.

FIG. 47 illustrates the high-level architecture of a system for operating a nanochannel device.

FIG. 48 illustrates the high-level architecture of a system that incorporates a nanochannel device into an integrated system for cellular packaging and functional testing of the long DNA products.

FIG. 49 shows a schematic diagram of the example nanochannel assembly device, monolithically fabricated to incorporate microchannels, linear nanochannel and adjacent Au electrode pairs.

FIG. 50 shows a fabrication process flow of the nanochannel assembly device.

FIG. 51 shows scanning electron microscope images (520 tilted, except panel b) of fabricated example nanochannel device with a pair of transverse Au electrodes near the single linear nanochannel and a pair of Au electrodes near the microchannel regions.

FIG. 52 shows scanning electron microscope images (52° tilted, except panel b) of fabricated example nanochannel device with 50 nm, 60 nm, 80 nm, 100 nm, and 200 nm linear nanochannels (D/W=depth and width of nanochannel) in parallel and a pair of Au electrodes near the microchannel regions.

FIG. 53 shows a schematic diagram of the bench-top test setup with electrical and fluidic control, and functional monitoring/testing.

FIG. 54 shows brightfield and fluorescence images of DNA loaded into the channels of an example nanochannel assembly device without and with applied voltage.

FIG. 55 illustrates the ligation of two DNA strands under control of an electrode.

### DETAILED DESCRIPTION

### Overview of the Detailed Description

A DNA assembly device is disclosed. The device includes a chip having at least one nanochannel integrated therein, the at least one nanochannel having a reaction zone where ends of at least two distinct DNA molecules can be maintained proximate to one another; and circuits and electrodes for controlling movement of the at least two distinct DNA molecules.

Additionally, a system is disclosed that includes first and second devices as disclosed herein, wherein each of the respective nanochannels are fluidically connected. Another system is disclosed

that includes a device as disclosed herein having multiple nanochannels. In this system, the device contains a network of nanochannels. In embodiments, the number of networked nanochannels is 1-10, or 10-100, or 100-1000, or 1000-10,000, or 10,000-100,000, or 100,000-1,000,000, or more than 1,000,000.

Additionally, a method of joining two DNA molecules is disclosed. The method involves introducing a first DNA molecule to a first nanochannel, wherein the first nanochannel contains a reaction zone; introducing a second DNA molecule strand to a second nanochannel, wherein the second nanochannel is also connected to the reaction zone; and electrically controlling the movement of the first and second DNA molecules strands to the reaction zone where the first and second nucleotide strands undergo a series of joining reactions.

### Definitions and Interpretation

As used herein, the term “DNA” in various contexts may refer to single stranded or double stranded forms of the molecule. The term “DNA” may also in various contexts refers not only to strands composed of the four bases A, C, G, T, but also of ribonucleotides such as in RNA, other base analogues, such as U (uracil), I (Inosine), and other well-known universal bases or base analogues or modified or marked bases, including well-known epigenetics marks on bases, such as 5mC (5-methyl-C), as well as dye-labelled bases, or bases modified for future labelling or conjugation, such as biotinylated bases, or thiolate bases, and in general any other widely known modified forms of bases used in DNA oligos, including possible modifications in the sugar or backbone of DNA as well. In addition, where it makes sense in context, the term DNA encompasses other nucleic acid (NA) polymers such as RNA (Ribo-), PNA (Peptide-), LNA (Locked-), and diverse forms of XNA (Xeno-).

As used herein, the terms “DNA assembly” or “joining DNA” refers to any process for physically connecting together two or more existing DNA molecule strands, with the connection at or near the ends, to produce a single molecule strand. In various contexts, such strands may be entirely single stranded DNA, or entirely double stranded DNA, or DNA that is partially single stranded and double stranded. Such strands that are assemble or joined may be connected by covalent phosphate backbone bonds, or may be joined through the hydrogen bonding of complementary regions, or in some contexts may be joined through other chemical reactions and chemical groups, such as carbon chain linkers from the end of one backbone to the start of another.

As used herein, the term “nanochannel” refers to any channel structure that is nanometer (nm) scale in its width and depth dimensions, such as up to 10 nm, or up to 100 nm, or several hundred nm, and substantially longer in its third dimension of length, such as 1000 nm or longer, 10,000 nm or longer, or 100,000 nm or longer, or up to 1 millimeter (mm) or longer, or 10 mm or longer. Such channels may be straight, curved, or branched in various contexts. Such channels may reside in a single plane, or may extend into 3D within a material substrate.

As used here in, “phosphoramidite synthesis” or “the phosphoramidite method” or “chemical synthesis” refer to any of the family of standard or well-known chemical cycles employed for synthesis using phosphoramidite bases, such as those used for commercial DNA oligo synthesis, or those deriving from the original methods such as put forth by Marvin Caruthers.

As used herein, the term “chip” refers to a semiconductor integrated circuit chip. In certain contexts where this is clear, it may refer to a CMOS chip.

As used herein, the term “CMOS”, which is an acronym for “complementary metal-oxide semiconductor”, and refers to chips that are made by the CMOS process.

As used herein, “DNA synthesis” refers to fabrication of physical DNA through a series of chemical reactions, in accordance with producing a desired specific target sequence or sequences.

As used herein, “DNA sequencing” refers to processes for reading the identities of the series of bases in a DNA strand or strands.

As used herein, the term “PCR”, which is an acronym for Polymerase Chain Reaction, generally refers to any means of amplifying or copying DNA, including by thermo-cycling PCR, or isothermal PCR reactions, or generally any other processes that can be used to amplify or copy DNA.

As used herein, the term “error correction” or “error correcting code” or ECC, refer to means of transforming a primary data string into another data string or strings such that various types of errors or corruptions of the source string be detected and corrected to recover the source string. In contexts where this makes sense, these may also refer to the process of using such encodings to correct errors. Many such methods are well-known for error correction for the transmission of binary strings, i.e., strings composed of the two symbols “0” and “1”. Many of such well known methods have versions that extend to 4 symbol strings, such as DNA sequences composed of the 4 symbols “A”, “C”, “G”, and “T”, or conversely two DNA letters may be used as binary string symbols, such that all binary ECC methods may apply directly. Any such methods are encompassed by use of this term, as they may apply in context.

### DETAILED DESCRIPTION OF ASPECTS AND EMBODIMENTS

Disclosed herein are compositions, devices, methods, and systems related to using nanochannels for the joining, or “assembly” of multiple shorter DNA molecules to form a single longer continuous DNA molecule.

In an aspect, a DNA assembly device is disclosed. The device includes a chip having at least one nanochannel integrated therein, the at least one nanochannel having a reaction zone where ends of at least two distinct DNA molecules can be maintained proximate to one another; and circuits and electrodes for controlling movement of the at least two distinct DNA molecules.

In embodiments, movement of the at least two DNA molecules is controlled electrically. In embodiments, the DNA molecules have lengths of about 50-about 1,000 bases, or about 1,000-about 10,000 bases, or about 10,000-about 100,000 bases, or about 100,000-about 1,000,000 bases, or about 1,000,000-about 10,000,000 bases, or more than about 10,000,000 bases. In certain embodiments, the chip contains multiple nanochannels, each of which can operate independently. In certain embodiments, the chip is a CMOS chip. In certain embodiments, the top of the at least one nanochannel is sealed. Various methods and implements can be used to seal the nanochannel.

In embodiments, the surfaces of the at least one nanochannel are functionalized. In embodiments, at least one portion of the at least one nanochannel is constricted. In certain embodiments, the reaction zone is fluidically connected to an end of the at least one nanochannel. This permits a DNA molecule from one nanochannel to interact with a DNA molecule from a second nanochannel. In certain embodiments, the reaction zone is configured along the length of the at least one nanochannel.

In another aspect, a system is disclosed that includes first and second devices as disclosed herein, wherein each of the respective nanochannels are fluidically connected.

In another aspect, a system is disclosed that includes a device as disclosed herein having multiple nanochannels. In this system, the device contains a network of nanochannels. In embodiments, the number of networked nanochannels is about 1-about 10, or about 10-about 100, or about 100-about 1000, or about 1000-about 10,000, or about 10,000-about 100,000, or about 100,000-about 1,000,000, or more than about 1,000,000. In embodiments, the chip is a CMOS chip.

In another aspect, a method of joining two DNA molecules is disclosed. The method involves introducing a first DNA molecule to a first nanochannel, wherein the first nanochannel contains a reaction zone; introducing a second DNA molecule strand to a second nanochannel, wherein the second nanochannel is also connected to the reaction zone; and electrically controlling the movement of the first and second DNA molecules strands to the reaction zone where the first and second nucleotide strands undergo a series of joining reactions. In certain embodiments, the first and second nanochannels are the same. In certain embodiments, each of the DNA molecules contain a mechanical blocking group. In certain embodiments, the method further involves removing the mechanical blocking groups following the joining of the first and second DNA molecules. In certain embodiments, the DNA molecules contain unique barcode elements for tracking each of the DNA molecules. The DNA molecules may have lengths of about 50-about 1,000 bases, or about 1,000-about 10,000 bases, or about 10,000
about 100,000 bases, or about 100,000-about 1,000,000 bases, or about 1,000,000-about 10,000,000 bases, or more than 10,000,000 bases.

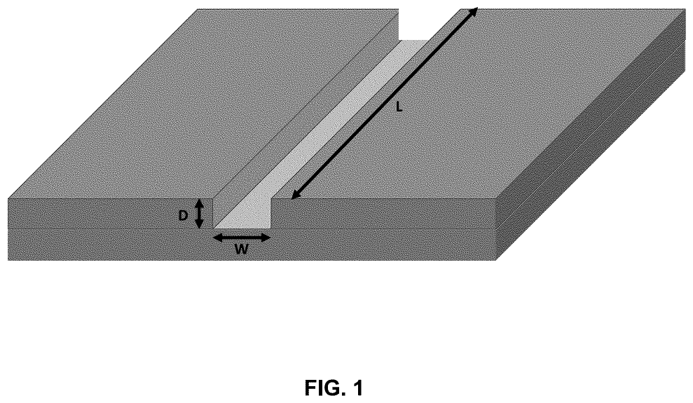

FIG. 1 shows a basic composition and geometry for a nanochannel. The channel is formed in a substrate material, and has critical dimensions of width (W), depth (D), and length (L). The width and depth are on the nanometer (nm) scale, such as less than 10 nm, or less than 100 nm, or less than several hundred nm. The length dimension is much longer than these lateral dimensions, such as up to 1000 nm, or up to 10,000 nm, or up to 100,000 nm, or up to 1,000,000 nm (=1 millimeter (mm)), or up to 10 mm or more.

For considering nanochannel dimensions that may be relevant for holding DNA molecules, it is noted that single stranded DNA molecules have a width of ˜1 nm, and double stranded DNA molecules have a width of ˜2 nm, and the length of such molecules, fully extended, is approximately 3.4 microns for every 1000 base pairs for double stranded DNA in a classical double helix (B conformation), or for single stranded DNA, 7.5 microns from every 1000 bases. Thus, for nanochannels to be of use for joining DNA, the channel width and depth must be >2 nm. The channel dimensions should also preferably be less than the persistence length of DNA, so as to discourage the DNA from balling-up in the channel, and thus in preferred embodiments would be <˜50 nm or <˜100 nm for dsDNA and <˜10 nm for ssDNA. In preferred embodiments, groups can be bound to the DNA, such as single-stranded-binding protein for ssDNA, and can increase the stiffness and persistence length of the DNA, enabling larger channel widths and depths, especially for ssDNA. The length of DNA dictates the channel lengths required for joining and manipulating DNA, and thus channels that in preferred embodiments may encompass the full length of molecules with 1000 bases to 1 million bases would imply channel lengths from several microns up to several millimeters, and similarly longer for even longer fragments.

The substrate material may be a semiconductor material, such as silicon or silicon oxide or quartz, or other semiconductors or semiconductor oxides, or metals or metal oxides, such as aluminum oxide, or a polymer or plastic material, or a glass or quartz, or a metal, or any material that would allow the fabrication of such a channel, many of which would be obvious to those skilled in nanofabrication. In preferred embodiments, the substrate material is an electrical insulator, so that voltages may be applied between the ends of the channel, without producing electrical currents in the substrate. In preferred embodiments, the material is silicon dioxide or aluminum oxide. Such channels may be formed, in preferred embodiment, by the standard methods of lithographic patterning, material deposition, and etching or liftoff processes.

Such channels may also have surfaces and sidewalls that are coated or functionalized to promote fluid flowing into the channels, such as treating the walls to make them hydrophilic, and also to prevent fouling or adhering of molecules to the wall. Such methods may include salinization, or other well-known coating methods that would be obvious based on this disclosure.

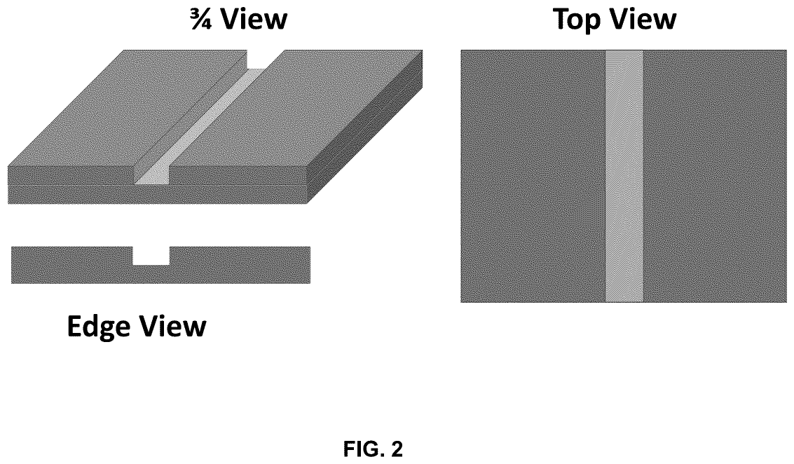

FIG. 2 shows other perspective views of such a nanochannel structure, which will be useful in illustrating the inventions of this disclosure.

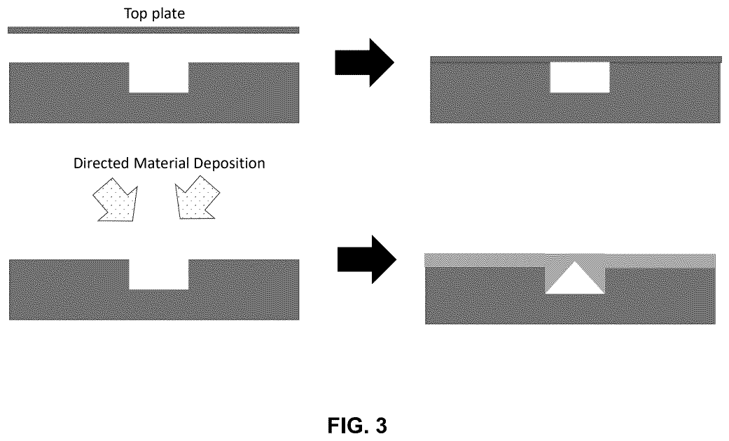

FIG. 3 shows that such nanochannels may further, in preferred embodiments, be sealed from the top, so that fluid may be enclosed in the channel, and thereby kept free from evaporation or contamination and otherwise protected from the environment. In preferred embodiments, as shown in the upper figure, a top plate may be placed on the substrate to form this cover, such as may be formed from a cover slip or thin silicon wafer or glass sheet, or plastic or polymer sheet, or many other cover materials that would be obvious based on this disclosure. In other preferred embodiments, such a top seal may be deposited by methods of material deposition, such as directional sputtering of material, that result in filling in as shown, ultimately sealing the top of the channel while leaving the channel largely open. Many such material sputtering or deposition processes would be obvious to those skilled in material science, such as directional sputtering of silicon oxide or aluminum oxide, or other oxide materials or electrically insulating materials. In preferred embodiments, the sealing material is a silicon or silicon dioxide or glass sheet, or directionally sputtered silicon or silicon dioxide.

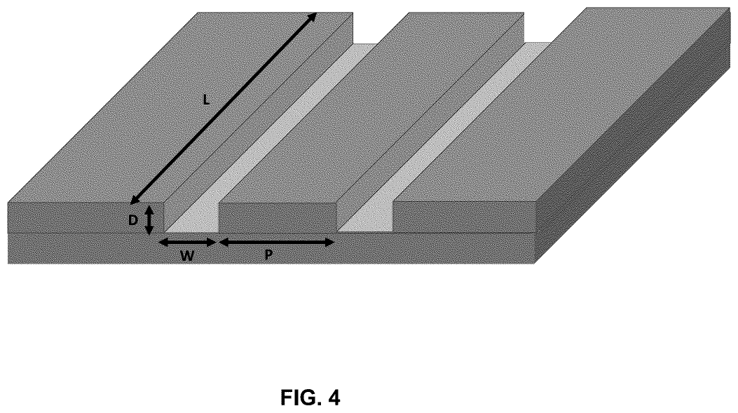

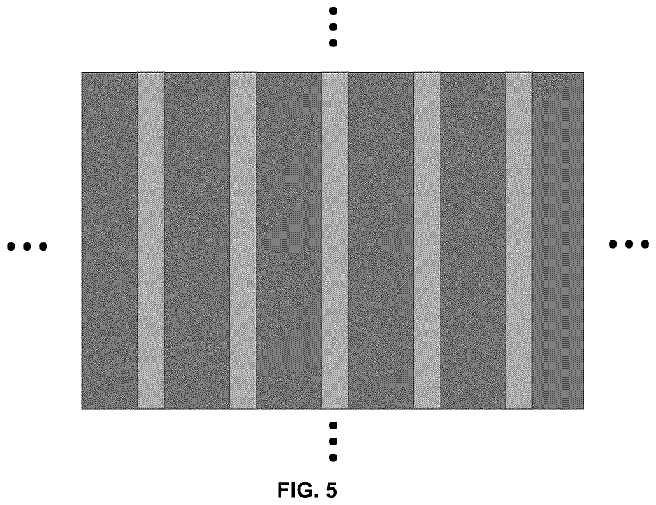

FIG. 4 shows two such nanochannels residing in parallel in a substrate. FIG. 5 shows that an unlimited number of such channels may be fabricated in a substrate, to support parallel operations of channels or more complex operations involving multiple channels.

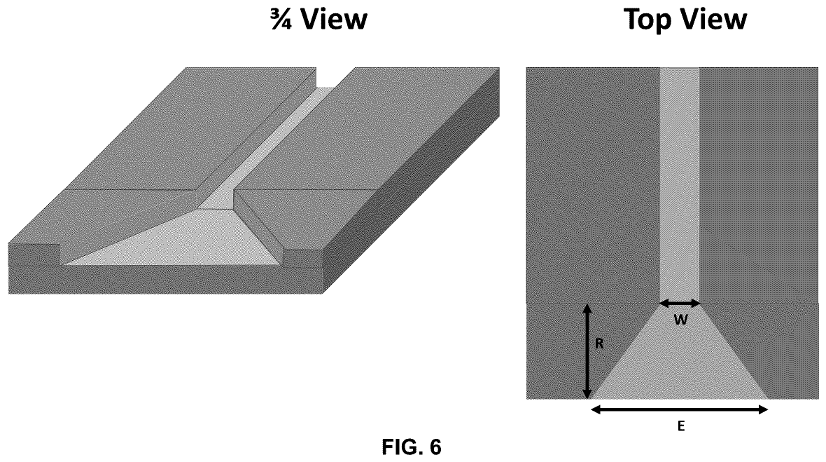

FIG. 6 shows that the inlet to a nanochannel in preferred embodiments may have a wider inlet that may taper to match the channels, to provide for a more effective transfer of fluids and molecules into the channel.

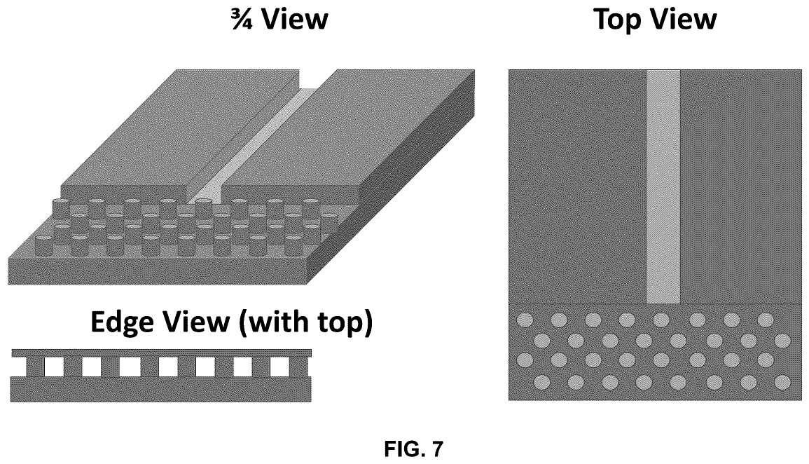

FIG. 7 shows that the inlet to the channel may also comprise an array of pillars, which serve to separate out, straighten out, and orient DNA molecules for entry into the channel. This provides an untangling function and orientation function that facilitates loading DNA into the channel. Such pillar arrays may have various pillar geometries, such as the circular cross section shown, or square cross sections.

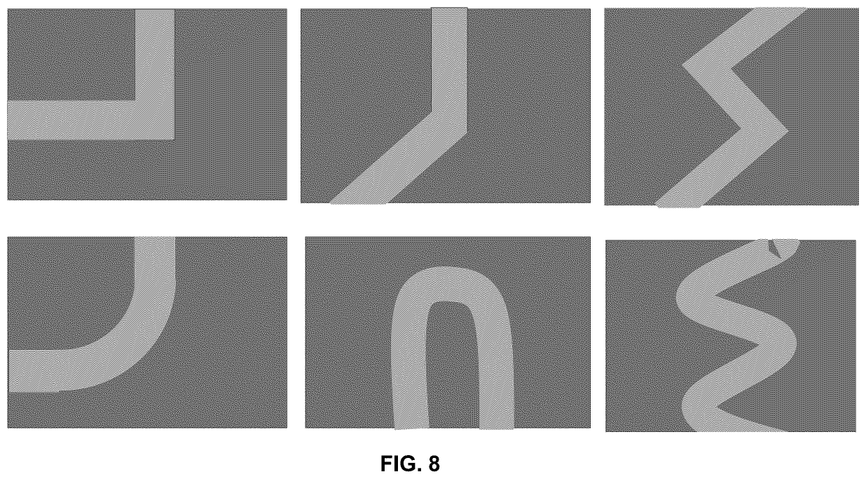

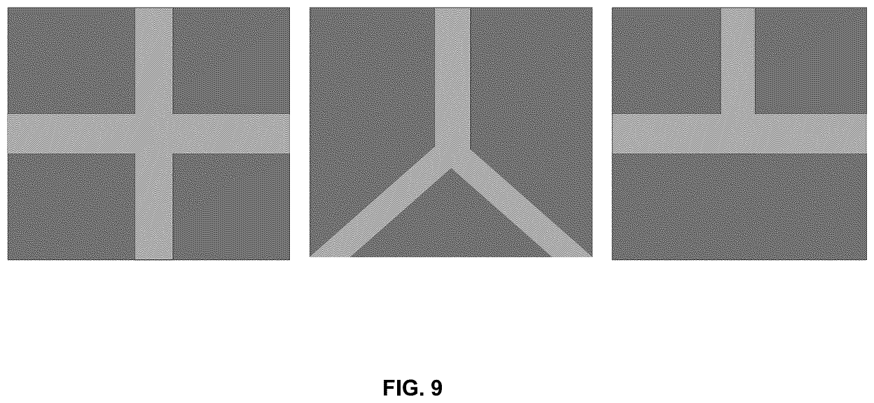

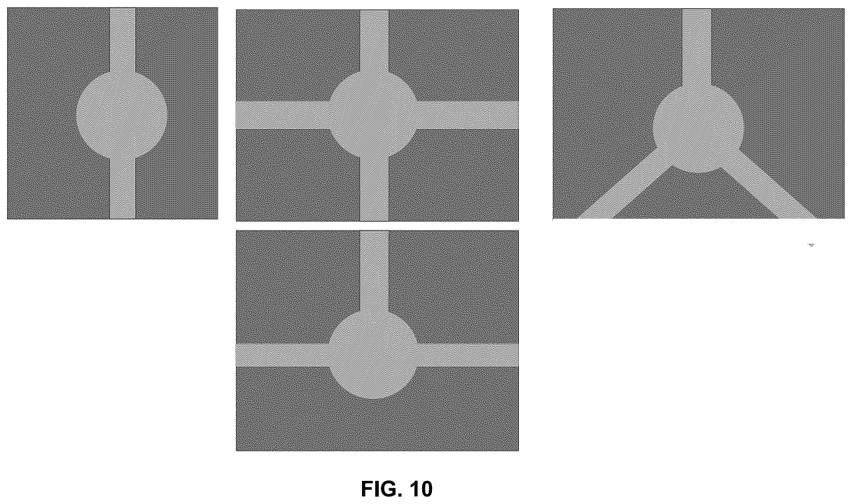

FIG. 8 shows that nanochannels may, in preferred embodiments, have curved or bent elements, to support various forms of motion or routing of molecules passing through the channels. FIG. 9 shows that such nanochannels may in preferred embodiments have junctions, where three or more channels join at one point. FIG. 10 shows that further, in preferred embodiments, the point of joining of the channels may be in an enlarged “reaction zone”, to facilitate the various types of DNA joining reactions that may be used.

In certain embodiments, the assembly reaction zones may comprise zones located at locations where incoming fragments for assembly enter the chip, such that in such zones, a multiplicity of an incoming sequence fragment, in the 100b to 20 kb size range, and preferably in the 1 kb-10 kb size range, are delivered to a reaction zone at an inlet to the chip, and a long fragment resident in a nanochannel in contact with this zone is positioned so that an end to be joined to the incoming fragment is in the reaction zone, and is allowed to potentially react with the multiplicity of fragments introduced to the zone. This may have the benefit of accelerating the reaction, due to the multiplicity of targets, and may also facilitate providing other reagents needed for assembly, such as enzymes, which may otherwise be limited in delivery if they must traverse an extended nanochannel to reach the reaction zone. In preferred embodiments, the incoming fragments will have a single reactive end, relative to the long partner for the reaction, so that only a single extension reaction may occur in the reaction zone, extending the long fragment from the nanochannel.

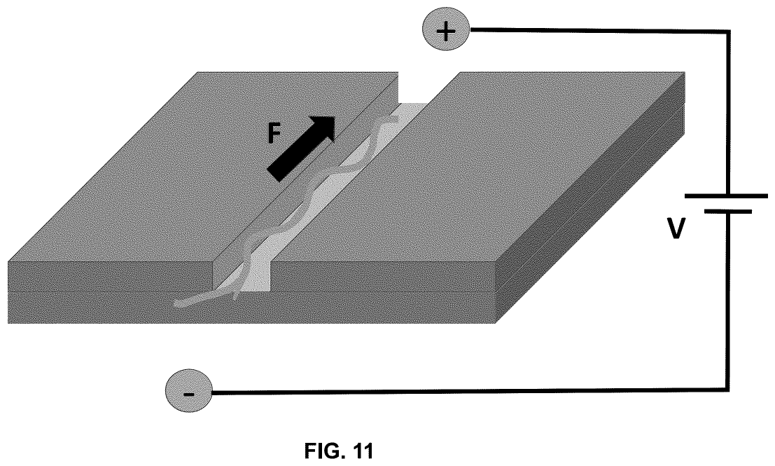

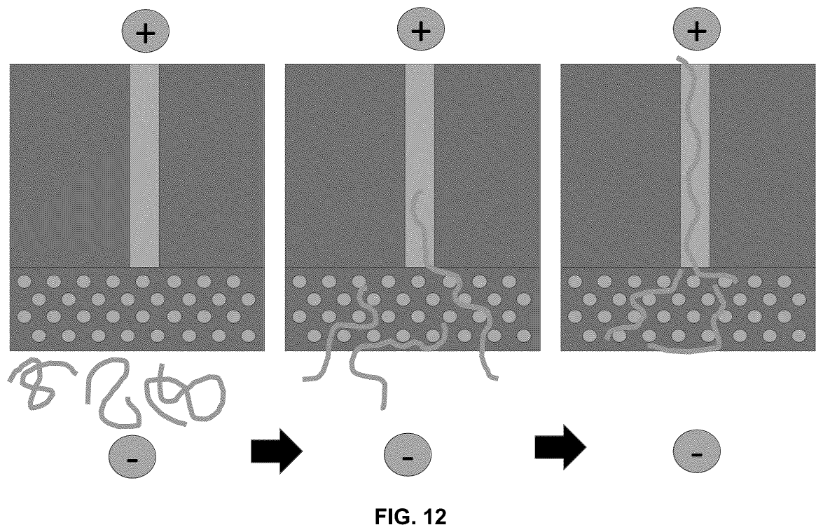

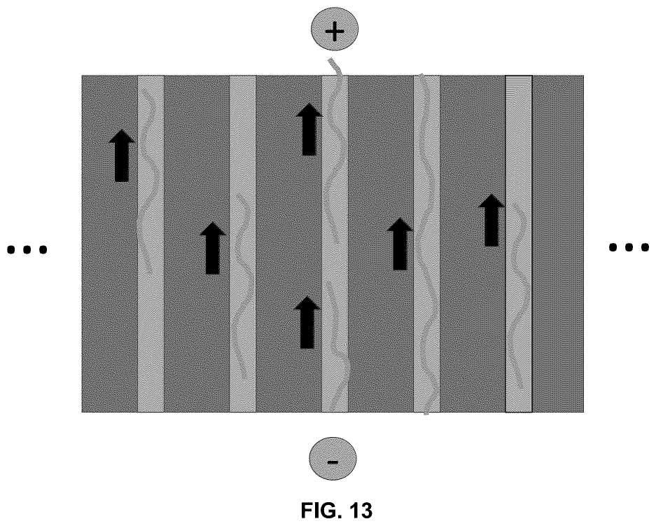

FIG. 11 shows how voltage is used to drive the translation of a DNA molecule through the channel, in solution. Since DNA is a negatively charged molecule in common aqueous solutions, in preferred embodiments a positive electrode attracts the DNA molecule in such a solution, causing it to stretch out into a linear, extended form in the channel, as it moves away from the negative electrode. In preferred embodiments, a top enclosure would be in place to prevent evaporation from the channel, and to confine the DNA in the channel and otherwise isolate it from the environment. Such a driving voltage may be maintained as a constant, may be varied in time, or may be turned off for a time. FIG. 12 shows how such a driving voltage will in practice attract DNA to enter the nanochannel, including the use of a pillar array and shaped inlet to facilitate long DNA entering the channel. FIG. 12 shows a time series progression, from left to right, wherein randomly coiled DNA is attracted to thread through the pillars, and sequentially enters the channel. FIG. 13 shows that many such channels could operate in parallel, as long as all are exposed to the driving voltages. In preferred embodiments, the solution may be common DNA buffers, or more dilute forms of these, with lower ionic strength, to reduce the screening of the applied electric field.

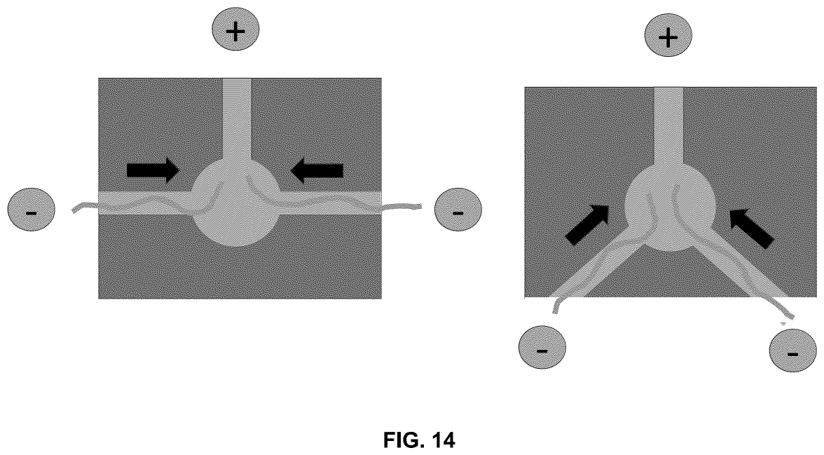

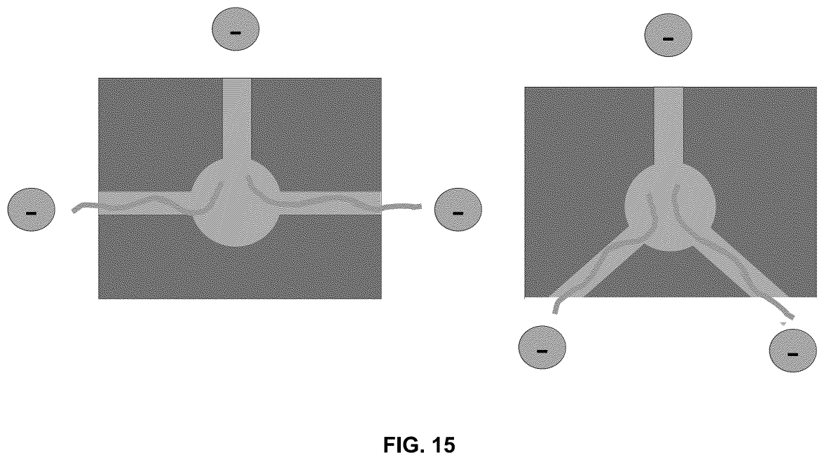

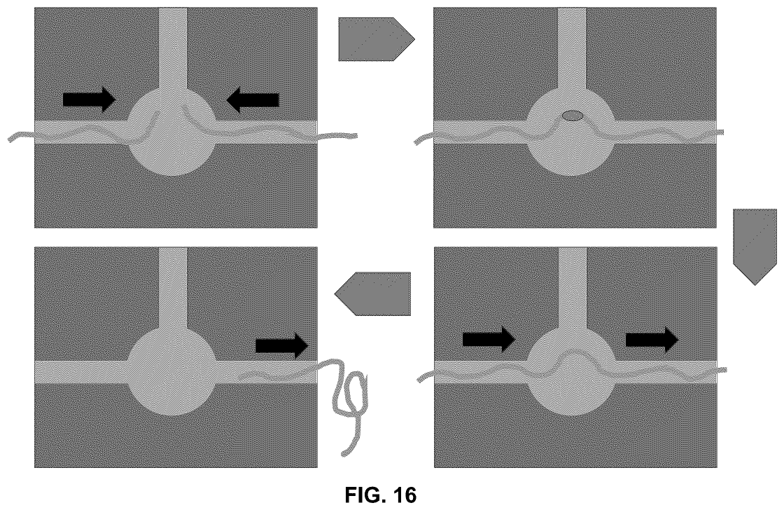

FIG. 14 shows preferred nanochannel embodiments for joining two DNA strands together. By application of a positive voltage on one channel, and negative voltages on the two that contain fragments, the ends of the fragments will be brought together in the reaction zone. As shown in FIG. 15, turning off the driving positive voltage at the appropriate time can pause the fragments with ends in the reaction zone. As shown in the time series of frames in FIG. 16, in preferred embodiments joining can be performed by first attracting the ends of the two DNA fragments into the reaction zone (upper left), then preforming a joining reaction (upper right), as indicted by the highlighted dot at the ends, then (lower right) the driving voltages can be set to attract the joined fragment back out of one inlet, and the fragment exits the zone into a nanochannel (lower left).

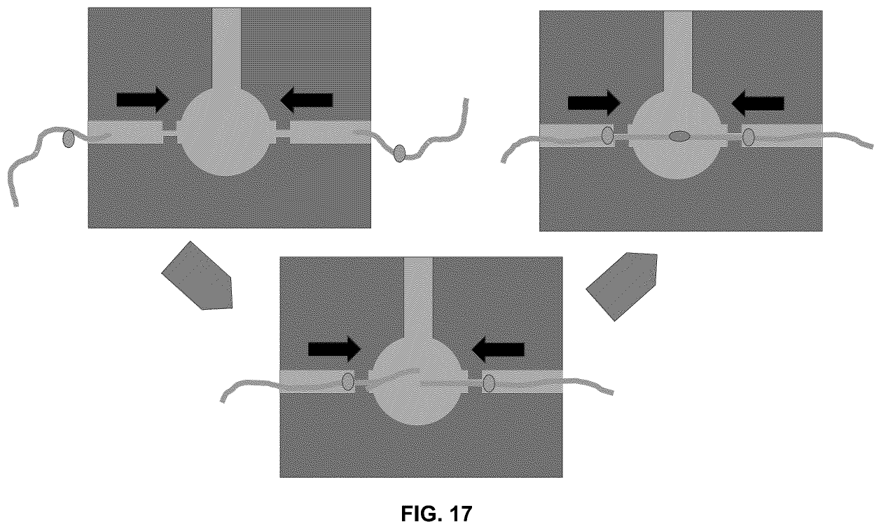

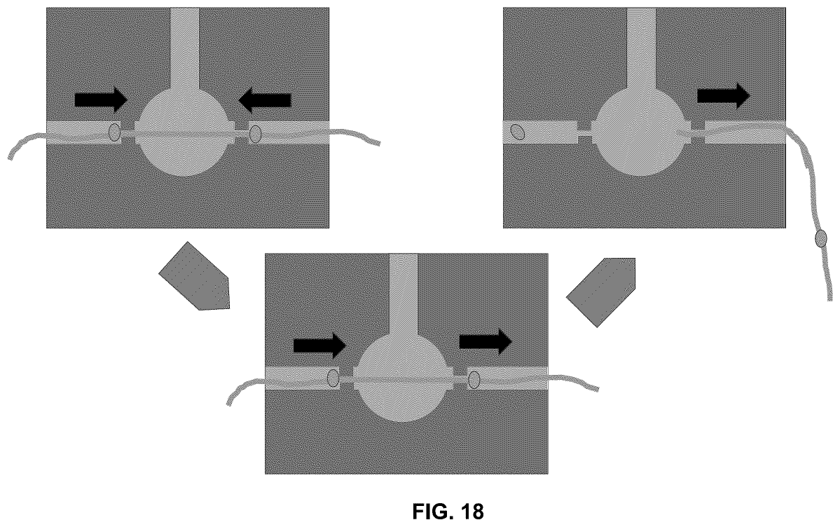

For the basic joining as shown in FIG. 14, the efficiency of the joining reaction strongly depends on the ends of the two fragments coming together in the right conformation for joining. In many reactions, this would require the ends to get within a few nanometers of each other, perhaps even within 1 nm of each other, depending on the joining chemistry. This may occur very rarely if the ends are not well positioned in the reaction zone, and thus the joining reaction may require a long time, or may not occur at all. Therefore, in preferred embodiments it is desirable to have a means of precisely positioning the ends of joining, such that they are much more likely to react. FIG. 17 shows a class of preferred embodiments for achieving this, wherein a use of “motion stops” positions the ends with much greater precision. As shown, a mechanical blocking group is specifically bound to the DNA a precise distance from the end of the fragment. Such a group in preferred embodiments, would be a particle in the size range of 2-50 nm, and preferably 5-30 nm, that has a specific DNA oligo on it, whose sequence complements a target sequence on the fragment to be joined, such that it hybridized with the DNA there, forming a removable blocking group, that is at a precise and specified location N bases from the end of the fragment. Similarly on the other molecule to be joined, such that, as shown, the reaction chamber channel entries have a constriction that is blocked by said blocking group, and thus the ends of the fragments will enter the reaction zone exactly up to the motion stopping groups. Such stop locations are defined so that the lengths of the segments that extend into the reaction zone can touch as needed, thereby greatly favoring the ends engaging in the joining reaction. As shown in FIGS. 17 and 18, the fragments enter, join, and then the channel driving force is aligned to force the joined fragment in one direction, with such force as to strip off the blocking group, so that the joined fragment may exit into one channel.

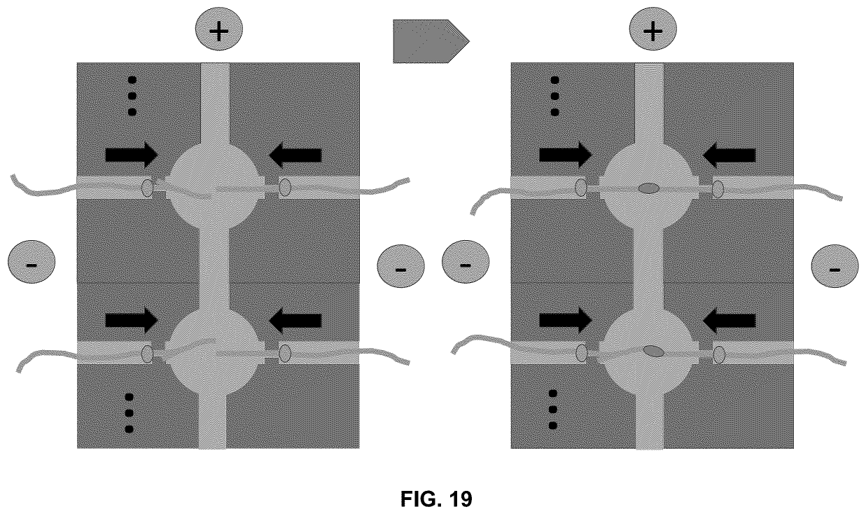

FIG. 19 shows that such a method using motion stops can be used in parallel, to join many pairs of fragments with the same sequence of controlling voltages, controlling the operation of multiple reaction zones. In the left frame, the reaction zones are loaded with fragments with motion stops, in parallel. In the right frame, the joining reaction occurs in parallel.

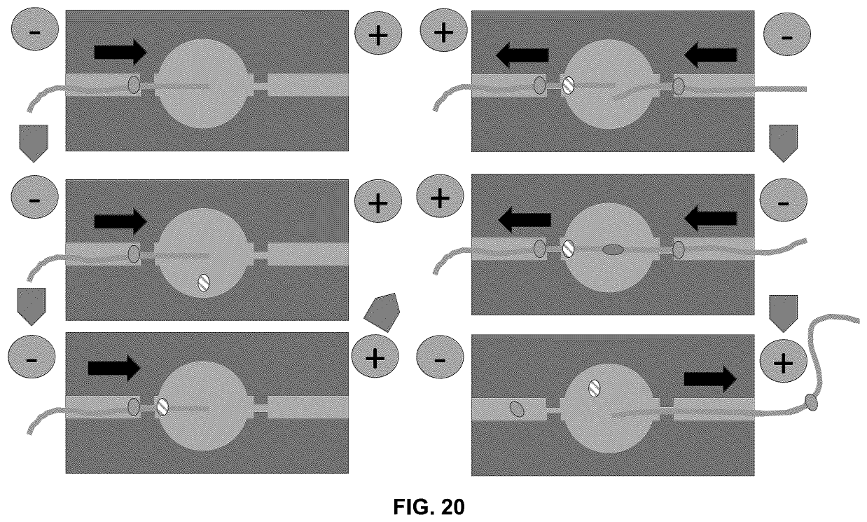

FIG. 20 shows another embodiment, in which a motion stop group is used in conjunction with a locking group, in order to achieve greater control over the joining process. FIG. 20 shows a time series of frames: in the first frame (upper left), a fragment with a stop group is loaded into the reaction zone. As shown in the second frame (med left), a locking group is introduced or resident in the reaction zone. This is a group that can bind the fragment so as to lock it in place, so that it cannot be readily pulled back out by an opposing driving force, as shown (lower left frame). In preferred embodiments, this group is also a nanoparticle attached to oligo that binds a complementary site on the fragment, to again form a removable group that prevents the fragment from backing out, which is bound in a precision defined location. Then, (upper right), the driving force in the channel is reversed to bring in a second fragment, with a motion stop group on it, such that the two fragment ends are in proximity to favor the joining reaction. The joining reaction occurs (mid right), as indicated by the highlighted dot, and finally (lower right) the driving field in the channel is reversed, to strip off all the removable blocking groups, so that the joined fragment exits into the channel.

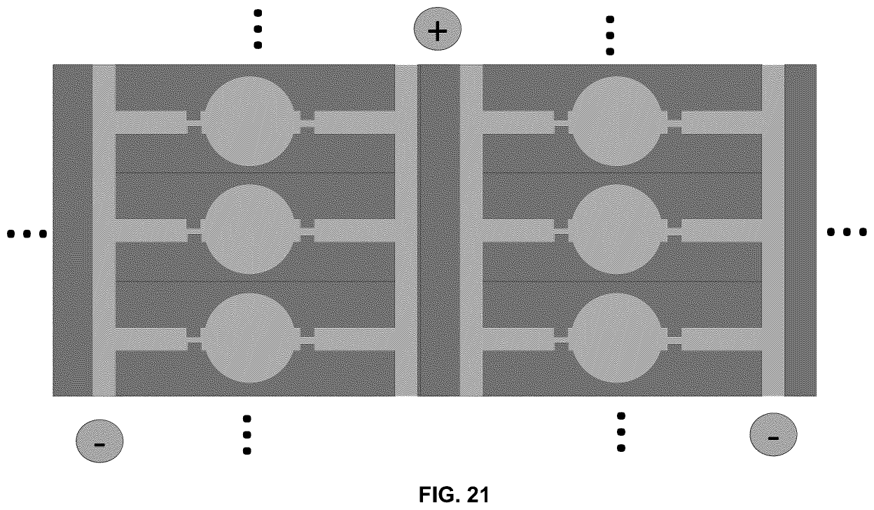

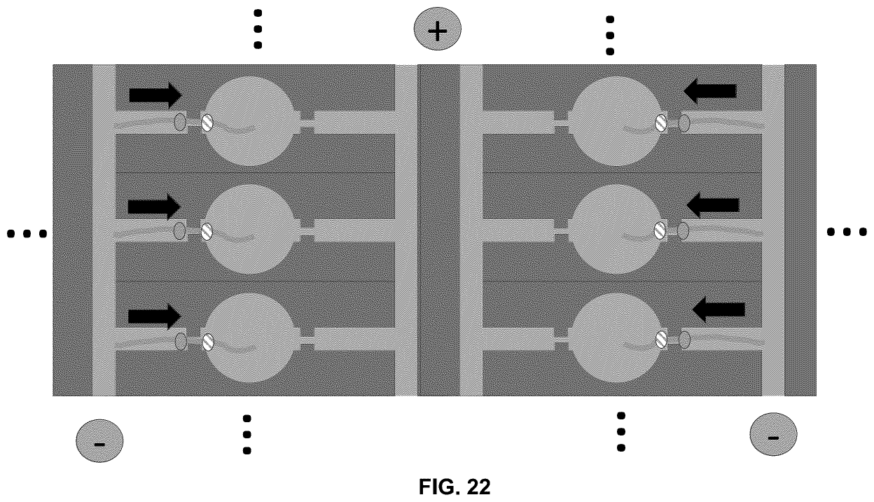

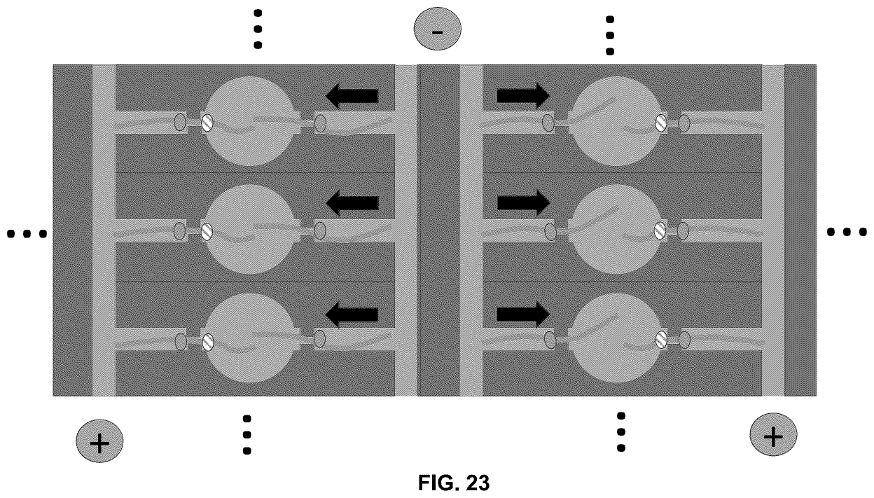

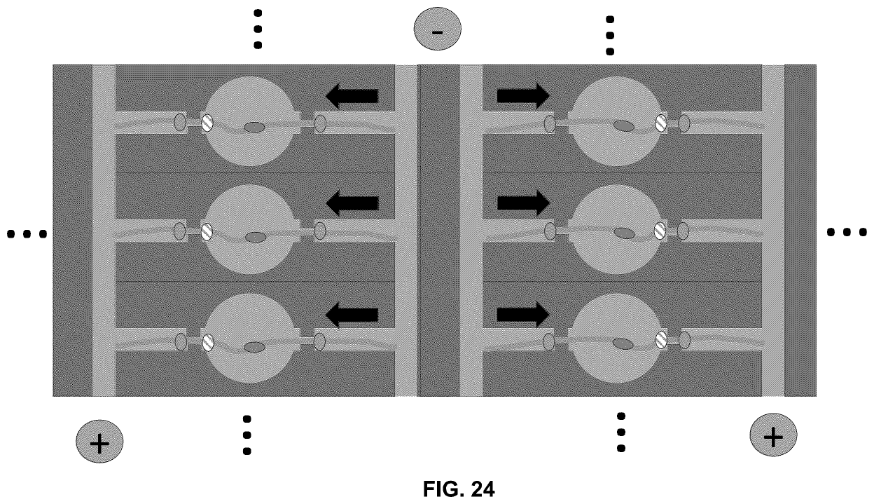

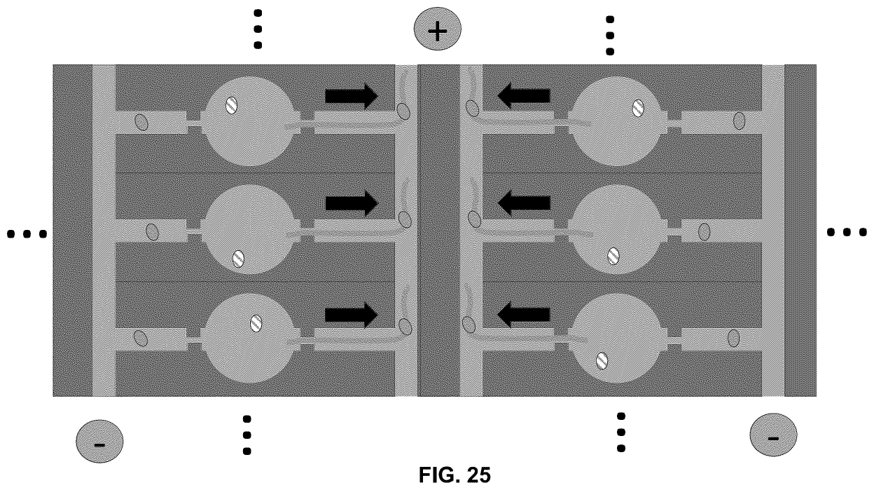

FIGS. 21 through 25 show a time series of frames that show how some joining with motion stops and locking groups can be performed in parallel, so that many such joins can be performed in parallel, with the same application of controlling voltages. This is a parallelization of the process represented in FIG. 20.

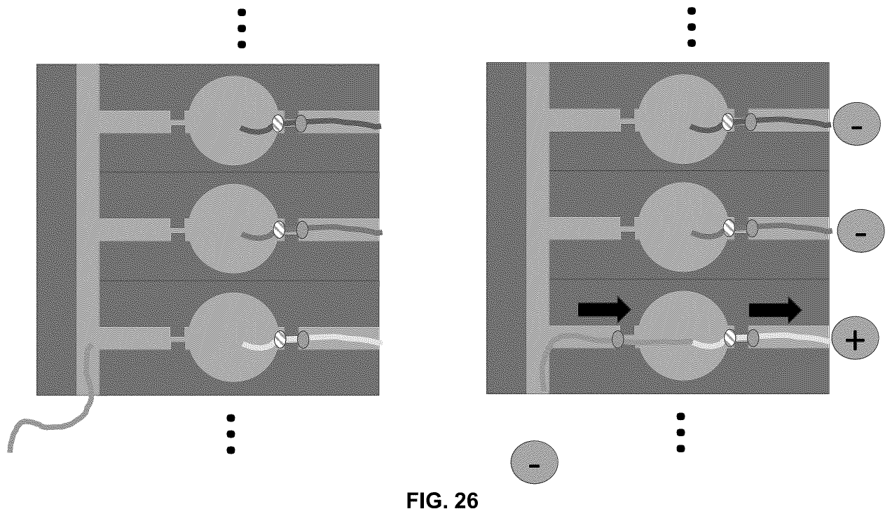

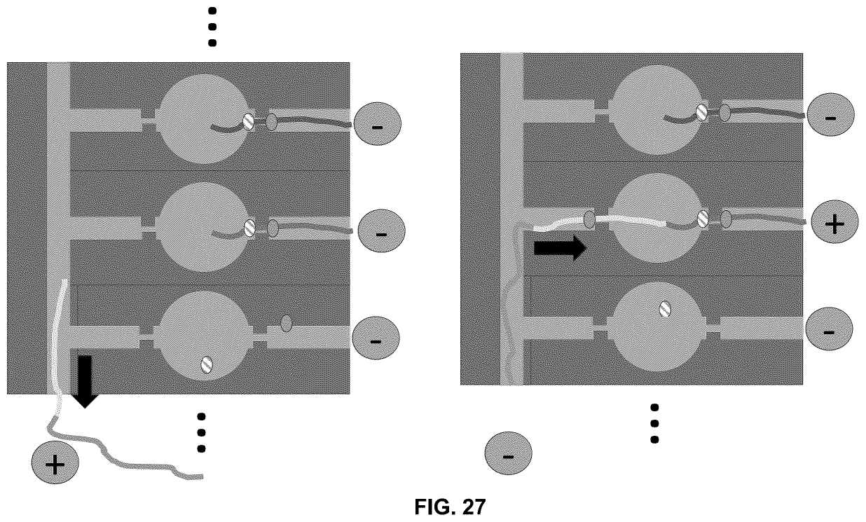

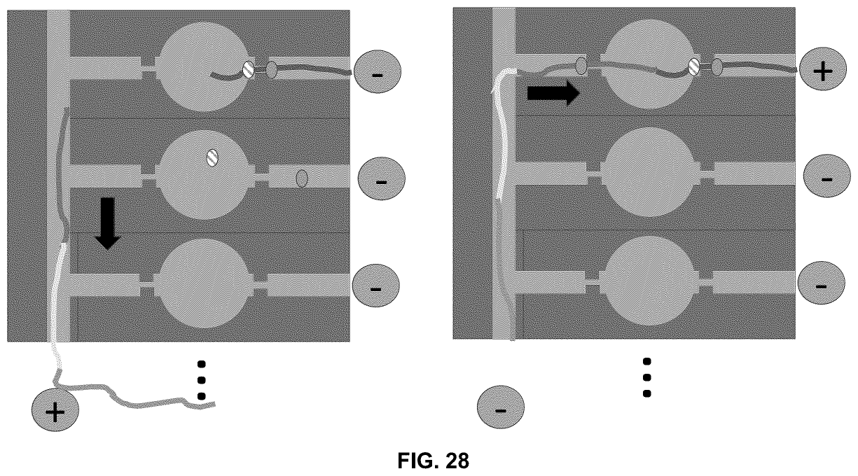

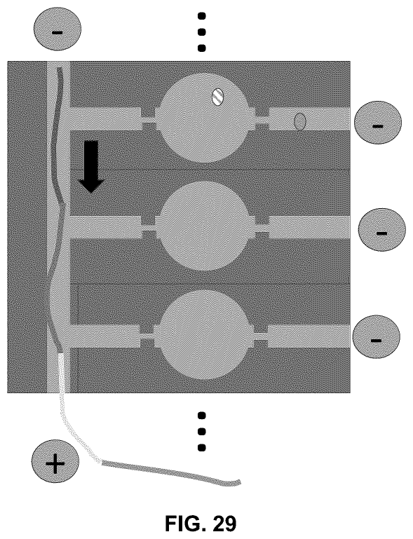

FIGS. 26 through 29 show a channel geometry and time series of frames for a method of serially joining DNA fragments onto the ends of a growing strand. At the start, in FIG. 26 left, motion stops and locking groups have been used to preload multiple fragments on the right side of the reaction chambers. A fragment to be extended is attracted in at the left to the first reaction zone shown, by applying a positive voltage to that zone as shown, and a motion stop is used to position it for joining reaction in the first chamber (right), where it achieves a join to the first of the serial fragments. In FIG. 27, left, the channel driving force is reversed to back this joined fragment out of the first reaction zone, and then (right) a positive voltage is applied to the second reaction zone, to again load the fragment into that zone and achieve a join to the second serial fragment. As shown in FIG. 28, the backup and attraction into the third zone is again achieved by applying the driving voltages as indicated, and activating the positive voltage on the third zone, where again the growing strand is attracted in and joined to the third serial fragment. Finally, in FIG. 29, the driving voltage is again reversed to back the DNA strand out of the third reaction zone. As indicated by the ellipses, this process can be continued without limit, to join any number of fragments serially. In preferred embodiments, N up to 10, up to 100, or up 1000 or more such serial joins may be performed, to produce a final fragment that is the original starting fragment joined with the N other fragments in series.

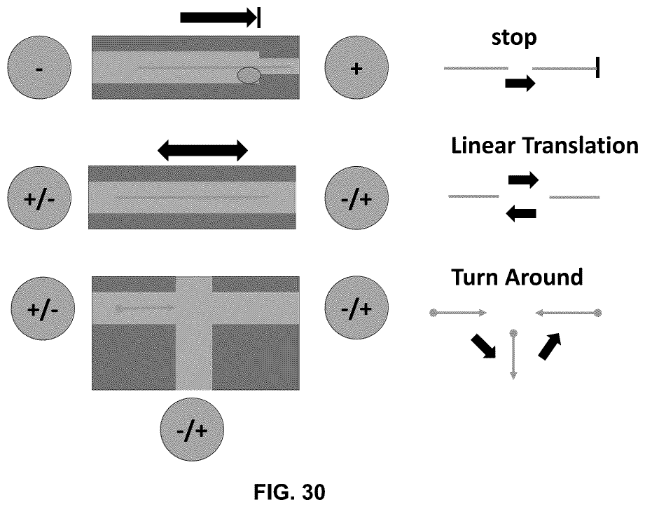

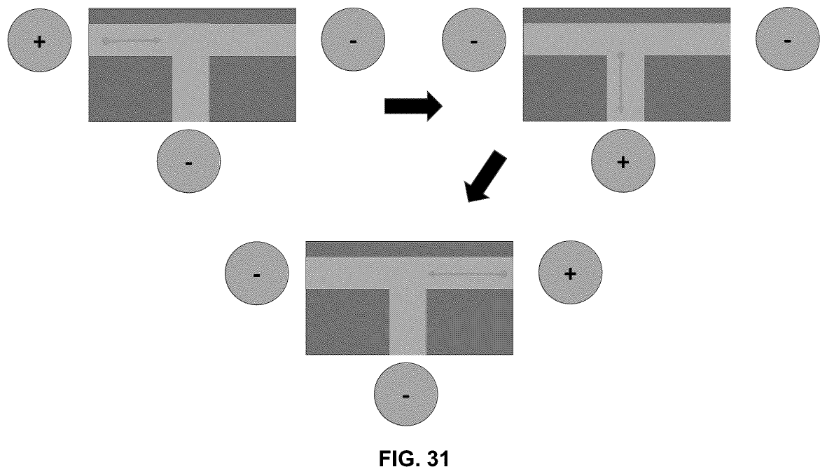

FIG. 30 shows basic elements of motion control that can be used within nanochannel devices. The upper panel shows the motion stop element, used to bring a fragment end to a defined location. The middle panel shows a channel with voltage control options that can be used to translate a fragment linearly in either direction. The lower panel shows a 3-channel junction that can be used to turn around a linear DNA fragment. Such a turning around element may be useful when the fragment is oriented to have a start and stop end, and it is desired to access a specific end. The method of turning around such an oriented fragment is shown in FIG. 31, here the time series goes from upper left, with fragment pointing left, to upper right, where the positive voltage attracts it down into the other branch, and then (lower) the positive voltage is moved to the right, pulling the fragment there, and achieving a right-directed orientation, thereby turning around the fragment from its original orientation.

### Input/Output

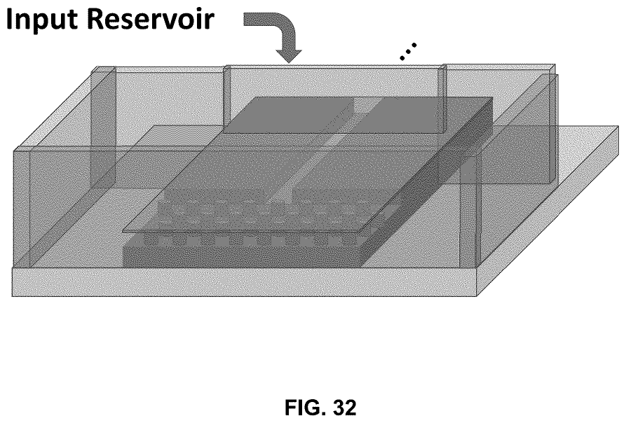

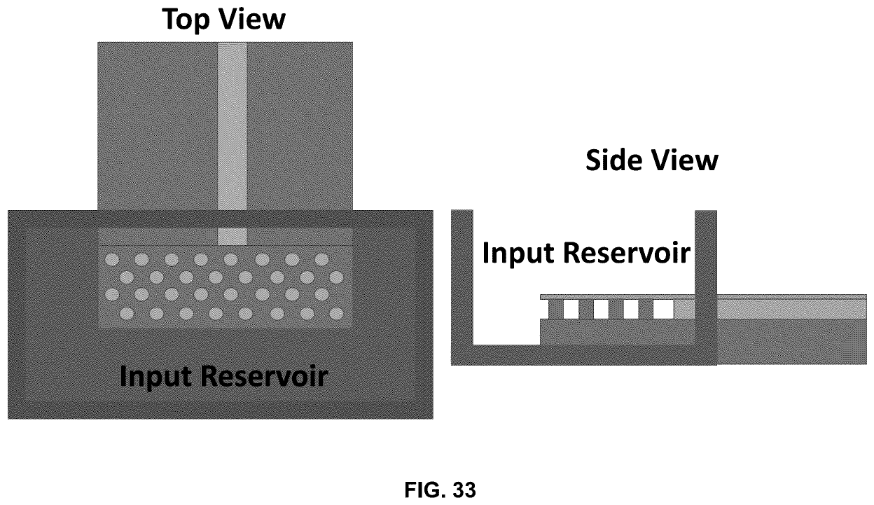

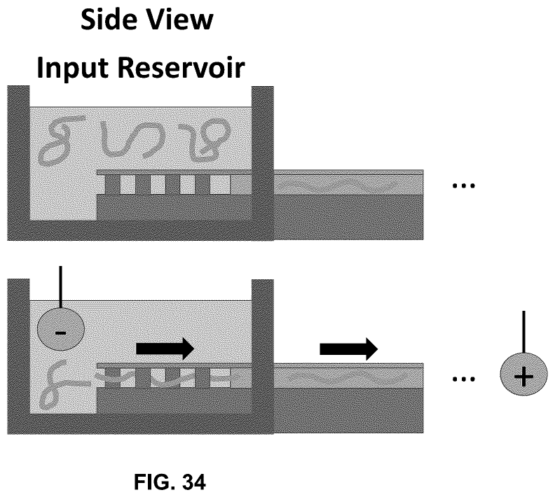

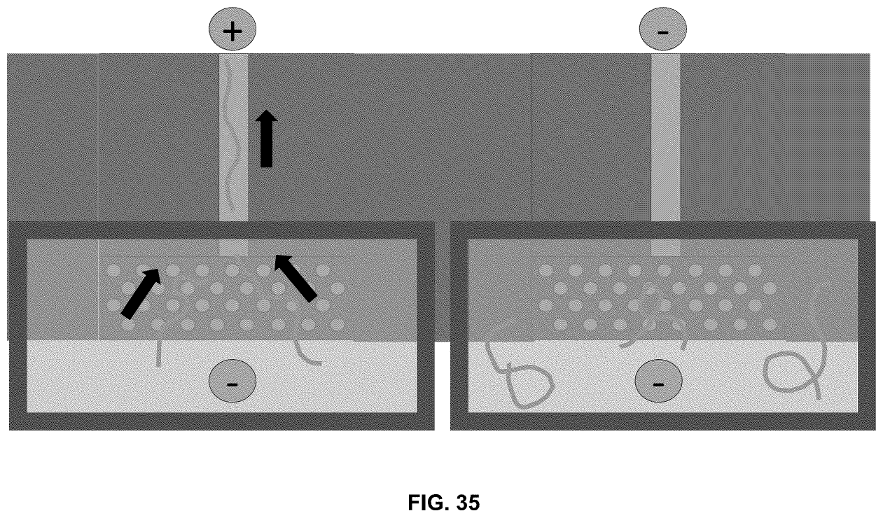

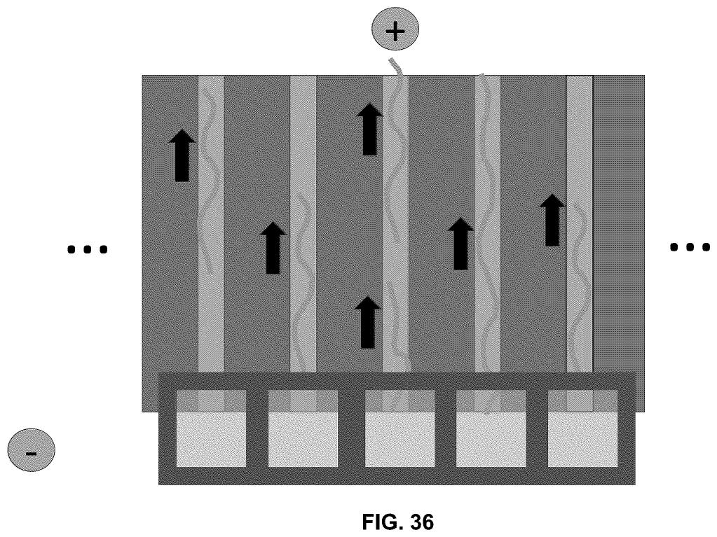

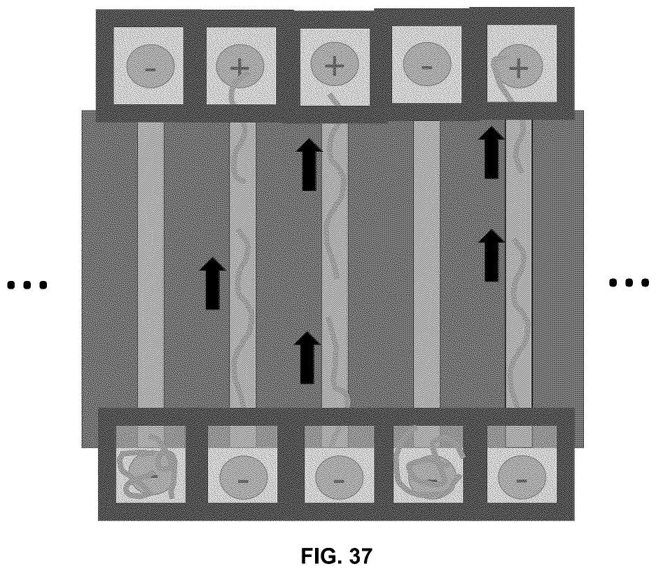

FIGS. 32 and 33 show a preferred embodiment of the full input reservoir for a nanochannel, which consists of a larger reservoir for fluid, interface to the pillar inlet to the channel. FIG. 32 shows a 3D view, and FIG. 33 shows top and side view depictions. FIG. 34 shows how such an inlet port loaded with DNA loads the nanochannel with input fragments when the driving voltage is applied to the channel. FIG. 35 shows a top view of this same process of loading DNA from the inlet port into the channel. Such a port under the reverse voltage also serves as an outlet port which is a repository for fragments that have been processed in the nanochannels. FIG. 36 shows a preferred embodiment of multiple inlet ports loading parallel nanochannels with fragments, under the action of common applied driving voltages. FIG. 37 shows how independent driving voltages can be applied to different channels, so that they can be selectively loaded from their input ports, based on the applied driving voltages.

### Nanochannel Device Architectures

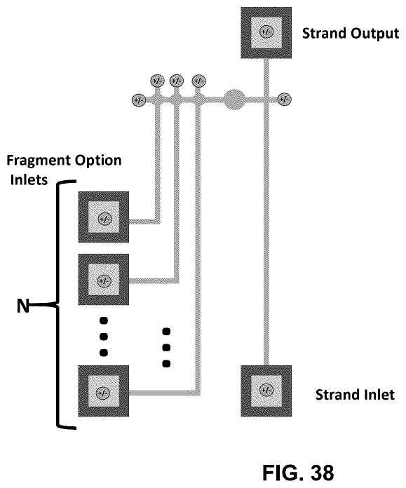

FIG. 38 shows a circuit-schematic view of a nanochannel device that takes an input strand from the inlet port, and joins to it one strand selectable under electronic control from N different optional fragments loaded into N inlet ports, and the resulting joined fragment is transferred to the outlet port. The various circuit details for the schematic are presented in the above disclosures, and so are represented here for brevity in the evident simple schematic form. Many functionally similar variations on this architecture are obvious based on the present disclosure, and all these are covered by the present disclosure.

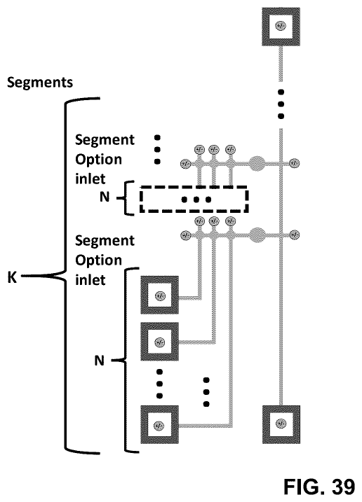

FIG. 39 shows a circuit schematic view of a nanochannel device that takes an input strand from the inlet port, and joins to it a series of K segments, where each segment is selectable under electronic control as one from N different optional fragments loaded into N inlet ports, and the resulting joined fragment is transferred to the outlet port. The various circuit details for the schematic are presented in the above disclosures, and so are represented here for brevity in the evident simple schematic form. Many functionally similar variations on this architecture are obvious based on the present disclosure, and all these are covered by the present disclosure. In general, the numbers can vary by segment, i.e., for segment i, there can be Ni different optional fragments, for i=1 . . . K. In this way, the resulting joined strand has the structural form of J=S0−S1−S2− . . . −SK, where S0 is the initial strand, and each Si is from the set of Ni options for segment i. There is a total of C=N1×N2× . . . ×Ni× . . . ×NK distinct possible constructs, and of which can be constructed by this circuit, and the circuit can construct and output any of these in series. For example, if there were K=10 segments, and each segment had 4 options, there would be a total of 410˜1 million different strands. Note that segments can be omitted as well, so there are really Ni+1 options for segment i, including omission of the segment.

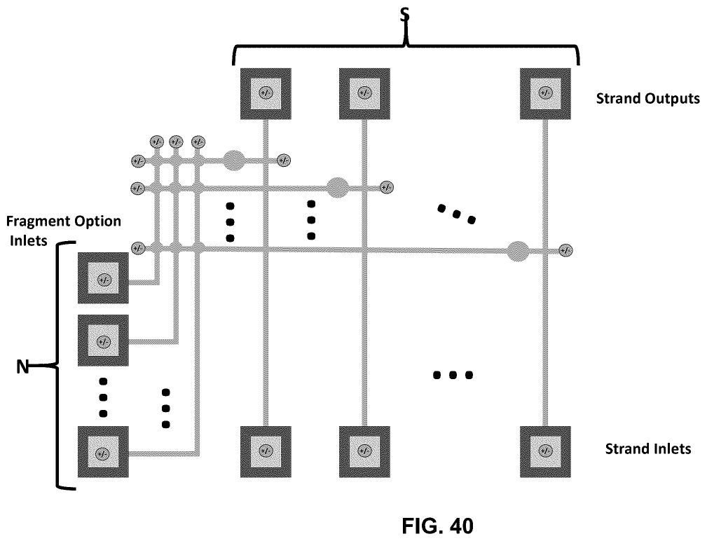

FIG. 40 shows an extension of the nanochannel device of FIG. 38, in which there are S total parallel joining channels, taking S inlet starting strands, and output S joined strands, and each such strand select to join one of the N optional fragments to the respective starting strand.

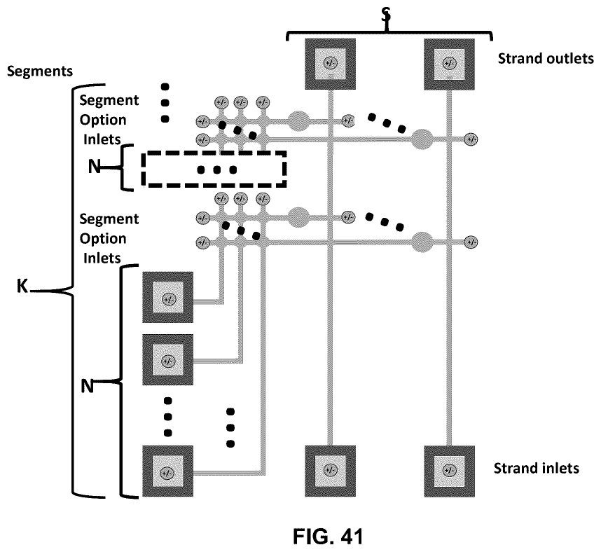

FIG. 41 shows an extension of this nanochannel circuit to support S parallel joining channels, taking S inlet starting strands, and output S joined strands, where each is composed of K segments, with segment Si chosen as desired from the Ni options. In the control of this circuit, if different output channels want different ith segments, those can be loaded into the respective reaction zones in parallel, whereas if two or more joining channels contend for the same segment, it has to be sequentially loaded into them, as they address the same segment inlet port.

Of note, many functionally similar variations on the disclosed architecture are obvious based on the present disclosure, and all of these are intended to be covered by the present disclosure.

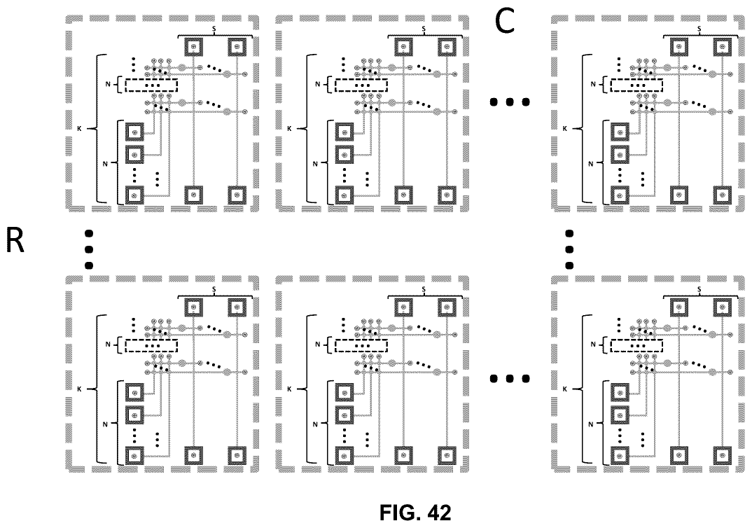

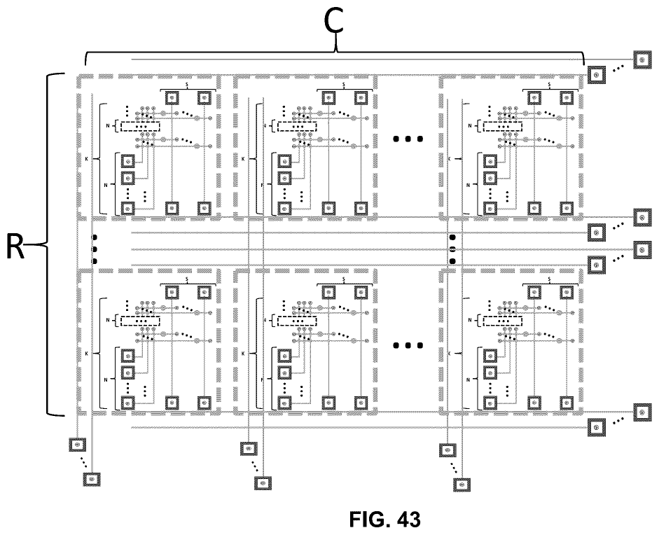

FIG. 42 shows an array structure, where an array of the devices of FIG. 41 are laid out on one device, with R rows and C columns of such devices. This provides for a scalable architecture to have many such devices. FIG. 43 further shows this device array with outer level inlet/outlet ports, indicated to support the loading of all the internal R×C×K×N inlet ports, and to support outputting off device all the R×C×S output ports.

### Nanochannel Monitoring

In various embodiments, it is desirable to be able to monitor and detect that DNA strands, or other particles, have entered various channels or are located at certain points within a channel. Ways to monitor this include dye labeling of the DNA fragment, and use of optical microscopy to detect the presence of the molecule in various channels. The ways to monitor also include incorporating electronic sensors in the nanochannel, such as impedance sensors, or impedance spectroscopy sensors that measure across the channel at a check point of interest. Such sensor may also be deployed at motion stop points, to detect the presence of the DNA, or of the stop molecule.

### Chip Implementation of Nanochannel Devices

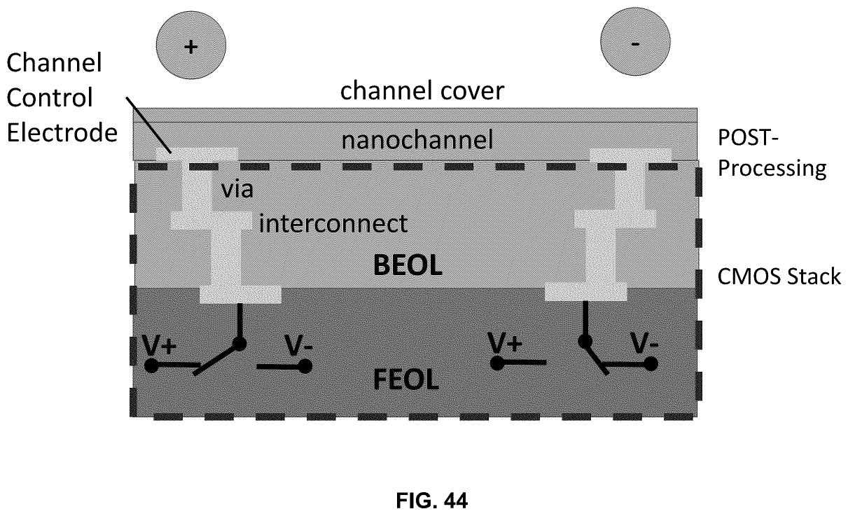

The nanochannel device architectures of FIGS. 38-43 are preferred embodiments implemented on semiconductor chips, and in particular in preferred embodiment, CMOS chips. FIGS. 38-41 show circuit-schematics for joining multiple fragments across multiple segments into multiple final strands. FIG. 42 shows how these circuit-schematics can be further parallelized, and FIG. 43 shows how the inlet and outlets can be externalized, if necessary. FIG. 44 discloses how to implement the critical control electrodes on CMOS chip devices. As shown, the nanochannel is post-processed on top of the standard CMOS stack, preferably using the same CMOS fabrication line and/or tools, so that the entire device could be made at one foundry, or perhaps in two foundries. The channel has fabricated in it control electrodes, and these are connected by vias and interconnects down through the Back-End-Of-The-Line (BEOL) stack, into the Front-End-Of-The-Line (FEOL) transistor layer, which implement switching circuits that allow the electrode to be set to the positive (V+) or negative (V−) control voltages. Using this basic method to deploy control electrodes in CMOS, the nanochannel devices can be preferably embodied in CMOS chip devices.

### Nanochannel DNA Assembly Systems

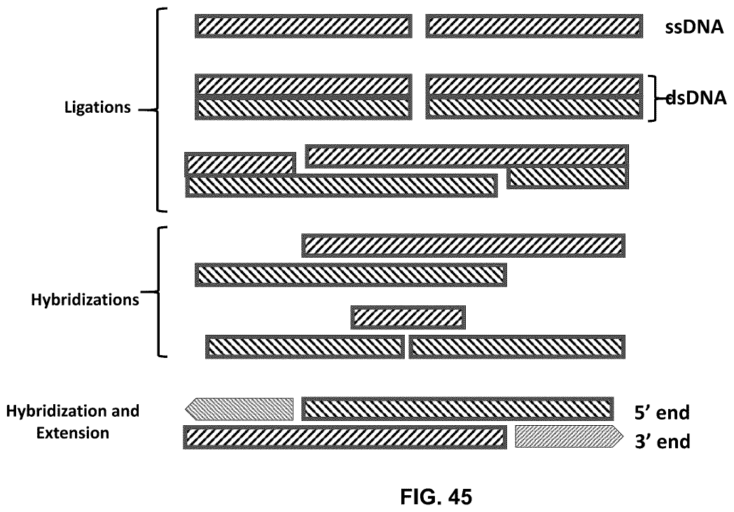

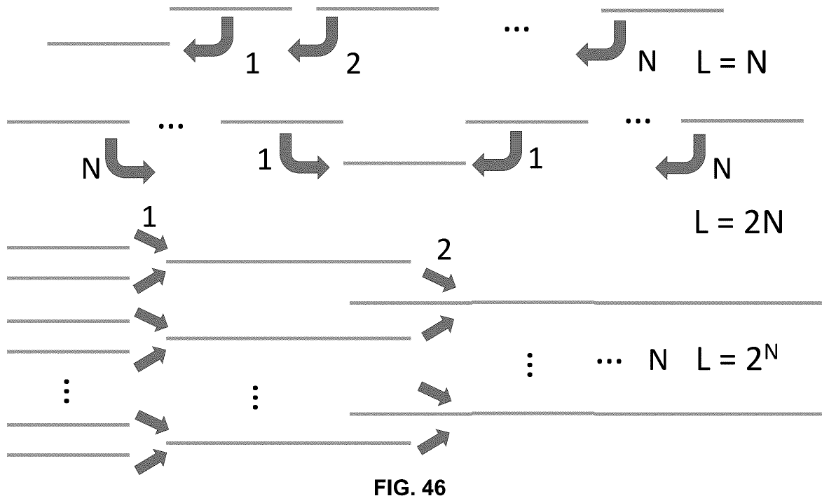

The basic nanochannel device or chips can be integrated into a system for the controlled production of long DNA fragments, built from various shorter building blocks in parallel. FIG. 45 shows various joining reactions that can be used to perform the joins: ligation of single or double stranded DNA, hybridization of DNA, or hybridization followed by enzymatic extension. Many others are obvious to those skilled in nucleic acid manipulation and are also intended to be covered by this disclosure. Various joining algorithms can be implemented in the nanochannel circuits, notably as shown in FIG. 46, serial joints of segment to one end, result in length L=N after N steps, or serial joining of fragment onto both ends, resulting in length L=2N after N steps (each step joins at both ends). Also, if fragments are joining in pairs, and again in pairs, and so on, the pieces double in length in each cycle, and after N cycles the fragment length is L=2N.

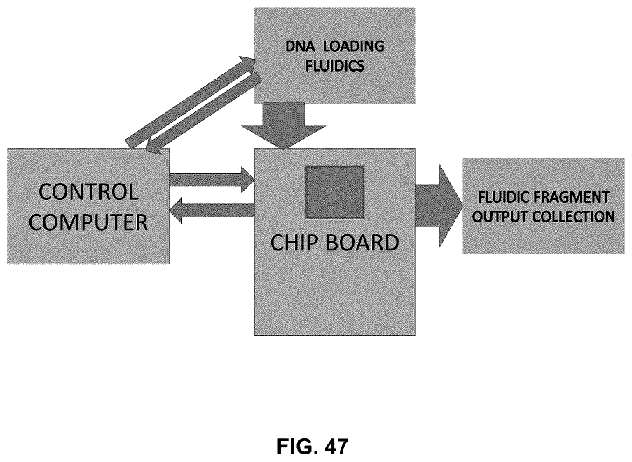

FIG. 47 discloses the high-level system architecture for a DNA assembly system, in which a control computer controls both the nanochannel chip device mounted on a motherboard, as well as an external loading system that can fluidically load the outer level input ports, and another fluidic system transfers from the outer level output port. Many variations would be obvious for the structure of this platform, based on the present disclosure and all such embodiments are covered by the present disclosure.

### Synthetic Biology Applications

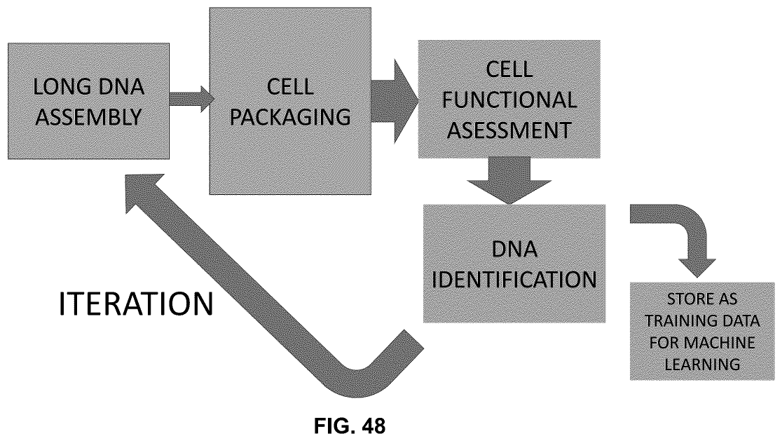

FIG. 48 discloses the high-level organization of a system to prepare long DNA fragments for synthetic biology applications. The long assembly system, as in FIG. 46, is transferred to a cell packaging facility, where they are packaged into cells, in preferred embodiments using electroporation, chemo-poration, or physical-poration methods. These cells carrying the DNA payloads in turn are put into a functional assessment to screen for desired phenotypic properties. Once such phenotypes are identified, the cells can be sequenced to recover the underlying DNA sequence. The identified sequences can also be put into a machine learning database, that keeps a growing record of what sequences produced which phenotypes. Ultimately, this supports machine learning, to eventually do machine learning assisted design. Many variations on this are obvious based on the presented concepts, and all these are intended to be incorporated within the present disclosure.

The nanochannel devices and methods disclosed herein, in certain embodiments, may further make use of these features to enhance performance, as detailed below.

### Motion Monitoring/Sensor Features

### Location Monitoring

In certain embodiments, the nanochannel device is provided with methods for monitoring, tracking or detecting the location of DNA or other useful particles in the channels. Such methods in preferred embodiments provide the ability to determine the location of one or both ends of the DNA molecule in a channel, at a point in time, or continuously in time, or to determine the time at which an end of DNA molecule arrives at or is present at some location, or to determine at a given point in the channel whether a DNA molecule is proximate or not. Such location monitoring can provide benefits for the assembly process, such as the ability to position the DNA strands with greater accuracy to facilitate their efficient assembly, to check or verify that assembly has occurred, and to track the growing strand through the assembly process, so as to be able to maintain the identity of a strand and at all times to know the current sequence of the strand under assembly. Methods that may be used for such location monitoring may encompass:

### Optical Monitoring of DNA in Channels

Optical monitoring may be performed using an external optical or imaging sensor, to detect an optical reporter signal originating from the DNA, or integrated optical sensors embedded in, or proximate to, the channel at one or multiple locations. The optical signal may arise from intrinsic fluorescence of the DNA, or from a stain or dye label applied to the DNA. This may include sequence specific labels, such as dye-labeled oligos that hybridize to the DNA strand, or optical barcodes based on sequence or sequence motif-specific binding of optical reporters. The means for achieving this encompasses many such alternatives and combinations for optical monitoring that are obvious to those skilled in the art of optical mapping of DNA, and microscopy of DNA, and optical sensors.

### Electrical Monitoring of DNA in Channels

Electrical monitoring may be performed using various types of electronic sensors that are embedded in or otherwise integrated into the channel at one or multiple points. Such sensors may encompass localized transverse impedance monitors, localized nanochannel constriction/nanopore ionic current sensors, or localize charge proximity sensors, or redox electrochemical sensors. The use of such sensors may be enabled or enhanced by the use of electronic labels or reports on the DNA strand, such as sequence specific or sequence motif-specific bound oligos providing electronic signature labels integrated into the oligo, such as charged groups, conductive groups, redox-active groups, polarizable groups, or size-exclusion groups, or groups that otherwise produce localized changes in conductance or capacitance. Such labelling may further encompass electronic barcoding for tracking of motion of fragments, through a spatial barcode pattern of signals along the DNA, or other electronic signatures that provide an identifying signature for tagging and tracing the specific identity of DNA strands. The means for achieving this may comprise many such alternatives and combinations for electronic monitoring that are obvious to those skilled in the art of electronic sensors.

### ID Tracking of Multiple DNA Strands

When multiple DNA strands are being processed in one or multiple nanochannels, it may be beneficial to be able to assign distinct identifiers to the strands, to enable tracking, distinguishing and determining the identities of the distinct strands at various times in the processing. Methods for this may comprise location-based identifiers, starting with knowing the location of a strand in a channel at a point in time, such as the point where the strand is introduced to a channel. This location-time stamp can become the identifier and may be affiliated with later location-time identifiers, to track strand identity. In other preferred embodiments, a spatial pattern of a label along the strand can provide a spatial barcode (such as using optical or electronic labels) on fragments, and this spatial barcode may be used to provide the unique identifier. Other means of creating a unique signature may be used, such as single labels that create a unique, repeatable signal signature in an electrical or optical sensor. The means for achieving this may comprise many such alternatives and combinations that are obvious to those skilled in the art of optical and electrical labeling methods.

As disclosed, any combination of parameters may be used to uniquely identify a strand/associate it with its point of origin or history throughout the assembly process on the chip, including overall length and mix of optical and electronic barcodes associated with each molecule. In some cases, the unique labels can be imparted by incorporation of modified or unnatural triphosphates during a step of templated enzymatic extension. Modified triphosphates may be used to impart all manner of the disclosed labels, including fluorophores, reactive handles for fluorophores and other post-synthetic derivatization. Triphosphates including these reactive handles may be functionalized with aminoallyl, azide, alkyne, thiol, alkene, amino, biotin, or desthiobiotin. It is preferred that the incorporation events are of relatively low probability relative to the natural incorporation events so that there are few modifications throughout a long strand, increasing the probability that the distance between the modifications provides a unique ‘code’ for each molecule that may help uniquely differentiate it. Generally, there is a degree of kinetic discrimination against incorporation of modified triphosphates relative to native ones, and generally the ratio of modified dNTP:natural dNTP in any given extension will be chosen to correct for this. In some embodiments, the ratio may be between 1:10, 1:100, 1:1,000, 1:10,1000, or 1:100,0000.

In other embodiments, strand labelling modifications may be imparted by binding another agent to a strand in a sequence specific manner. This may include proteins which have preferential binding motifs or designed nucleic acid probes which can bind as a triplex or invade a dsDNA duplex in a programmable manner, such as sequences capable of binding certain motifs through Hoogstein paring or PNA probes. In some embodiments, the presence of the binding molecule may be sufficient for labelling, as the additional bulk may provide an increased level of current deflection through a channel or pore. As before, the binding molecule may itself contain another label or a functional handle for post-synthetic installation of such a label. An advantage of this mode of labelling is that it is non-covalent and potentially reversible with change of local conditions such as pH or temperature.

Either mode of labelling may be performed ‘on-chip’ throughout various points of the process or in bulk reactions prior to the loading material on the chip. Some embodiments, particularly those utilizing bulk labelling, may include an optional step of filtering strands which are not uniquely identifiable relative to other strands in the ensemble of an assembly reaction. Filtering may be accomplished, for example, by selective use of potential to eject unidentifiable constructs from channels so that they may be used as substrates for further labelling or end up as waste.

### Assembly Monitoring

When performing a process to assemble strands in a nanochannel, it may be beneficial to have means to assess when a join has occurred, or to assess whether an attempt at joining was successful. Such monitoring to assess whether two strands have physically joined may encompass electrical or optical detection, in conjunction with a test method that provides a determination of the join having occurred or not. In one preferred embodiment, the methods comprise using forces applied so as to pull the strands apart, such as pulling on one end, or in opposing directions on the two strands, and using the location detection sensors to determine if the two strands move in a highly correlated way or not under such a test, thereby indicating they are joined, or not. In another preferred embodiment, the method encompasses making a strand length measurement, to assess whether the strand length corresponds to the joined length, or not. Such a method may comprise pulling one end to move the strand into a process that performs a length measurement of the strand, based on either detecting locations of the ends, or measuring a transit time past a sensor, and using the transit time or strand velocity/mobility to determine if a join has occurred or not. In another preferred embodiment, the method may comprise the use of detectable barcodes on each fragment, assessing whether the molecule has the combined barcode of the two fragments or not.

### Assembly Error Detection and Correction Methods

When performing a process to assemble strands in a nanochannel, it may be beneficial to have methods to detect assembly errors, and to correct such errors. In preferred embodiments, the error detection may comprise measurement of a DNA strand length or length-related feature such as mobility or velocity, or a barcode, or other feature of the strand that indicates an incorrect assembly via not matching to the expected properties of the intended assembled strand. In preferred embodiments, the corrective methods could comprise discarding failed assemblies, by moving the residual fragments to a disposal site, or may comprise retrying the assembly, such as in the case of a failed join.

### Motion Control

To assemble DNA strands in a nanochannel, it is beneficial to have means to control the motion of DNA strands in the channels. Such controls may be beneficial for bringing the ends to distinct strands together with precision for efficient joining reactions, for the routing of fragments through networks of channels, to stage fragments as needed to carry out a specified series of joins, and for checking whether joins have occurred through coordinated strand motion and detection, and for the error detection and correction process on the assembled strands as indicated above. Motion control may comprise the use of passive controls on motion and position, as well as active controls based on a feedback loop coordinating input readings from the strand position monitoring sensors, and from the strand identity monitoring sensors, all in conjunction with application of the driving forces to actuate motions, which could be fluidic or electrical.

### Passive Motion Controls

Passive motion control comprises applying motion driving forces to a strand, in combination with physical structures that result in the strand coming into an equilibrium position at a defined location, or with a strand end at a defined location. This does not require a sensor to provide any information on the strand location, that is determined by the equilibrium between the driving force, and the stop restricting further motion when engaged with the strand. Methods for such passive control may comprise physical motion stops or barriers that restrict further strand motion once the strand or strand end has contacted the stop or barrier. Methods for producing such a stop may comprise adding a large physical side group to the strand, which hits against a stop obstruction extending into the channel, thereby creating a localized restriction in channel width or cross section. Or such groups may be pinned within a trap, such as a localized indentation extending into the side of the channel, that is capable of catching the group. Such a group could in preferred embodiments comprise a magnetic or charged group that is held via magnetic or electrical forces. Such stops may be located at the end or ends of the strands, to thereby control the motion or portion of the strand end. In preferred embodiments such groups may comprise a DNA oligo hybridized to a single-stranded segment of the DNA strand, where the oligo may itself be the stop group (protruding from the primary single stranded DNA strand) or may carry on it a group that acts as the stop group, such as a bulky molecule or nanoparticle attached to the oligo. In preferred embodiments such stop groups may comprise a DNA hairpin, secondary structure, or knot at or near the end of the strand, acting as the stop. Such secondary structure stops provide the benefit that they may be displaced or dislodged by applying a sufficiently strong pulling force on the DNA. Stops at one end may serve the purpose of controlling the motion and location of that end, or they may be used to fix the location of that end, for the purpose of controlling the motion or position the other free end, under a force that extends the DNA strand in the channel.

The active motion control may comprise taking inputs from the location monitoring sensors, and use these sensor inputs in control logic that turns on or off the driving forces for strand motion or length extension (stretching), or maintaining position, so as to bring the strand or one or more ends of the strand to desired locations, at desired times.

Electrically controlled motion, whether passive or active, can be used to move DNA through the nanochannels (using DC or AC methods), or to precisely localize DNA at a point within a nanochannel (using DC or AC methods).

### Mechanical Preparation/Conditioning of DNA Strands

For purposes of motion and location control, it may be beneficial to prepare or condition the DNA strand to provide it with more favorable mechanical properties. In preferred embodiments, such conditioning may comprise means to make the strand more stiff, rigid and straight, or increase its mechanical persistence length, relative to the properties of the native (ssDNA or dsDNA) form of the strand. Means for stiffening the strand may comprise coating it with ssDNA binding proteins, dsDNA binding proteins, or other chemical agents that bind to or intercalate the DNA, or making more of the DNA strand into the dsDNA form rather than ssDNA form, by binding it to complementary DNA strands, or DNA analog strands that may even further enhance stiffness (such as PNA or LNA strands). In other preferred embodiments, the conditioning of the DNA strand mechanical properties may comprise altering the solution medium containing the DNA, to include gel matrix material, to increase the solution viscosity, or to include other matrix materials or reagents that prevents long DNA fragments from breaking or tangling. Many such means of stiffening DNA or reducing breakages or tangles are obvious to those skilled in the art of nucleic acid biochemistry.

In other embodiments, particles could be added to the DNA strand, which may have specific properties to assist in the assembly process. The particles may be at or near the terminus of one end, where the other end is undergoing the assembly process or otherwise available for enzymatic reactions. The particles may serve a number of purposes throughout the disclosed assembly procedures, including acting as additional means of controlling DNA mobility in inlets or through channels, wherein the size and nature of the particle may promote or discourage the entry of a DNA strand into a channel, or stop passage at particular points within the chip as detailed above. Particles may also act as solid-phase anchors during fluidic exchange as various buffers and reagents are passed through the channels, provide additional types of barcoding, where the particle size, fluorescence, or other property aids in strand identification, or otherwise alter the electrokinetic properties of a strand. The response of a strand-particle conjugate in response to applied AC or DC field may be greater or less than that of the strand alone, depending on the particulars of the strand size, particle size, and particle material. The particles may be comprised of any material typically associated with nanoparticles or microspheres to skilled artisans and can include polystyrene spheres, coated iron oxide particles, silica particles metal nanoparticles, quantum dots, diamond or carbon spheres, proteins or large complexes proteins, or resins commonly used for affinity chromatography. The nature of the conjugation between particle and DNA strand for assembly may be covalent or non-covalent, though in general non-covalent conjugates are easier to reverse. A preferred mode of reversible conjugation is hybridization, where a particle is coated with a capture oligonucleotide sequence at least partially complementary to the DNA strand undergoing assembly. In some embodiments, the duplex DNA strand can undergo hybridization if it is prepared with ‘sticky ends’, such as those generated by exonucleolytic digestion of one strand at its termini. Other embodiments may instead rely upon use of strands on the particles which are capable of binding to duplexed DNA in a sequence specific way, either by forming triplexes or by invasion of the duplex itself. There are diverse methods of reversing the conjugation once it has been formed including temperature, pH, applied electric potential, or by performing any chemistry which may cleave the particle bound complement from the particle itself. The precise chemistry depends upon the linkage between the capture oligonucleotide but there are diverse linkers and cleavage methodologies known to skilled artisans. Methods compatible with reuse of the particle are generally preferred.

It is generally preferred that a given particle is bound to only a single strand for DNA assembly at a time and aspects of the invention may include methods for associating particles and assembly strands in a 1:1 ratio. In some cases, assembly strands may be loaded into the channels prior to association with a particle, then repositioned so that a termini is protruding from an accessible region of the channel, where a particle may be positioned in close proximity to capture the assembly strand. In some embodiments, the particles may reside on one face of a nanochannel array like that shown in FIG. 43, and the assembly strands are introduced into the array and transported to the side of the array bearing the particles, while in other embodiments the strands are introduced to the array and particles are instead brought to the edge of the array bearing the desired strand termini. One aspect of these 1:1 assembly procedures is that they allow for further techniques which may improve the ease of ‘matching’ assembly strands with appropriate capture strands on particles. The sequence specific nature of capture requires at least some degree of designed complementarity between the assembly strands and the capture strands. In some embodiments, all assembly strands bear a common sequence element, or limited set of sequence elements, near at least one of their termini which are complementary to the capture strands on the particle. These sequence elements may be of a form which allows them to be removed in a global processing step, such as a restriction digest, at some relatively late stage of the assembly process. In other embodiments, the particles bear multiple capture sequences, in particular those representing the designed termini of the assembly strands. A set of N assembly strands may have up to 4N unique termini, and thus it may be desirable to attach a library of complements targeting binding to all such 4N termini. For reasonably small N, it remains likely that an assembly strand will encounter a suitable binding partner on the particle when they are kept in close proximity. The specific chip design, throughput, and nature of the particle all play some role in determining the preferred configuration of particles and capture strand libraries.

### Assembly Organization and Workflows

There are diverse assembly strategies and workflows which are compatible with the disclosed chip designs and capabilities. In some embodiments, assembly strands are loaded into the chip, like that depicted in FIG. 43 in a prescribed order, determined prior to the initiation of assembly. This has the advantage of allowing the assembly to be conducted in a pre-programmed way with minimal alterations to the process. In other embodiments, strands are labelled in bulk as disclosed above, just prior to or just after loading into an array of channels and identified by the combination of uniquely identifiable traits. The precise assembly algorithm, comprising the steps of motion and joining, is then determined in response to the molecules identified in the chip. One version of this design may utilize multiple capture and release steps of the termini from particles in order to anchor them while the needed reagent exchanges are conducted. Strand tracking and adjustments to the assembly program may be conducted periodically depending on the accuracy needed and intrinsic error rate of the process. In a preferred workflow, the size of the strands and design of their termini is appropriate for Gibson Assembly like procedures. In this embodiment, strands may be labelled in bulk, loaded into the chip with the reagents needed to perform such and are identified. The strands are then moved so that the intended termini can meet in a reservoir, the specifics of the motion program determined in real time. When the intended strands meet, they may be joined according to their designed complementarity, and the newly formed sequence can be moved on to join with the next desired termini. This allows for a near real time assembly process and reduces or eliminates the need for many steps of fluid exchange, strand immobilization and release, or both. The sequence specificity to the end joining operations also introduces a degree of error tolerance in the motion of the strands so that it is far less likely that two strands will join if they inadvertently collide in the channel array.

It is beneficial for a nanochannel assembly device to have a means for the Input of the DNA strands for processing and Output of the post processing resulting strands. These constitute the primary I/O capabilities of the device. In preferred embodiments, the input allows for many component DNA pieces to be entered into the channels. The incoming fragments may be oligos, with lengths as short as 25 or 50 bases, or more preferably fragments that are at least 100 to 500 bases, or at least 1000 bases, or 5000 bases or 10,000 bases, or 20,000 to 50,000 bases. In preferred embodiments, multiple identical fragments representing the component sequences of interest are transported into the nanochannels at multiple channel inlets, and the number of such inlet channels may be 1, or up to 10, or 100, or 1000, or 10,000, or 100,000 or 1,000,000 inlet channels. Incoming solutions carrying the different sequences may interface to 1 or multiple channels, up to 10, up to 100, or up to 1000 channels being fed by the same incoming solution of DNA fragments, with single molecules from this pool entering various of the multiple channels. In preferred embodiments, incoming pools may be transported by microfluidic lines or lanes interfacing to the 1 or more channels, or as droplets moving on an electrowetting digital microfluidics array to provide the droplets of DNA fragments to interface to the nanochannels.

The output DNA fragments in preferred embodiments will pass from nanochannels into holding channels, or isolation chambers or wells of a well plate, such as a 96-well plate or 384-well plate, or into droplets that are moved in a droplet microfluidic system, such as an electrowetting digital microfluidics system, or into the lanes or lines of a nanofluidic or microfluidic system. In preferred embodiments, the outputs media and physical format may comprise solutions or material matrices, such as gels, or processes and geometries that that avoid breaking long DNA as they exit the device.

In certain embodiments, the output of DNA strands may interface, via microfluidics, to other microfluidics chips for further processing.

### Additional Aspects and Embodiments

Without limiting any of the foregoing or anything else detailed herein, the present disclosure discloses nanochannel devices with measuring sensors for checking and tracking assembly of DNA molecules. In an aspect, CMOS-Chip-based versions of nanochannel assembly devices are discloses. In an aspect, methods for assembling DNA molecules using such devices are disclosed. Additionally, methods for serial addition of segments to growing DNA strands are disclosed. Additionally, methods for parallel assembly of DNA strands are disclosed. Additionally, massively parallel nanochannel systems for DNA assembly are disclosed. Additionally, use of passive and active stops for precision motion control during assembly are disclosed. Additionally, nanochannel assembly devices and methods that comprise specific elements of function are disclosed. Additionally, barcoding and sensor-based tracking of fragments passing through the disclosed systems is contemplated. Input DNA molecules may have lengths of about 50, about 100, about 500, about 1000, about 2000, about 5000, about 10,000, about 20,000, about 50,000, or well above 50,000 bases, as has been provided for herein.

In certain embodiments, the number of joins may be about 1, about 10, about 100, about 1000, about 10,000, to about 100,000 or more joins. Further, the number of parallel strands processed on a device may be about 1, about 10, about 100, about 1000, about 10,000, about 100,000, or about 1 million or more.

In an aspect, a system is disclosed that includes first and second devices as disclosed herein, wherein each of the respective nanochannels are fluidically connected. In another aspect, a system is disclosed that includes a device as disclosed herein having multiple nanochannels. In this system, the device contains a network of nanochannels. In embodiments, the number of networked nanochannels is 1-10, or 10-100, or 100-1000, or 1000-10,000, or 10,000-100,000, or 100,000-1,000,000, or more than 1,000,000. In embodiments, the chip is a CMOS chip. The system may include the nanochannel(s) being equipped with measuring sensors for tracking the DNA molecules and/or DNA assembly. In certain embodiments, the chip disclosed herein is integrated into a system for the synthesis and assembly of DNA.

### EXAMPLES

### Example 1. Fabrication of Nanochannel Assembly Device

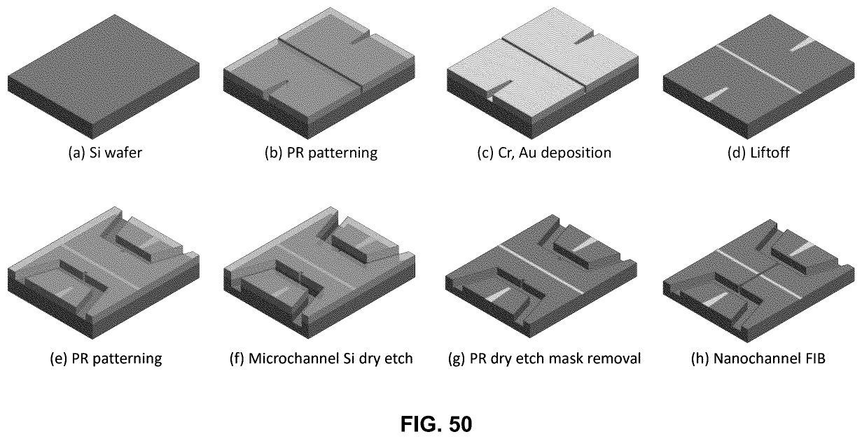

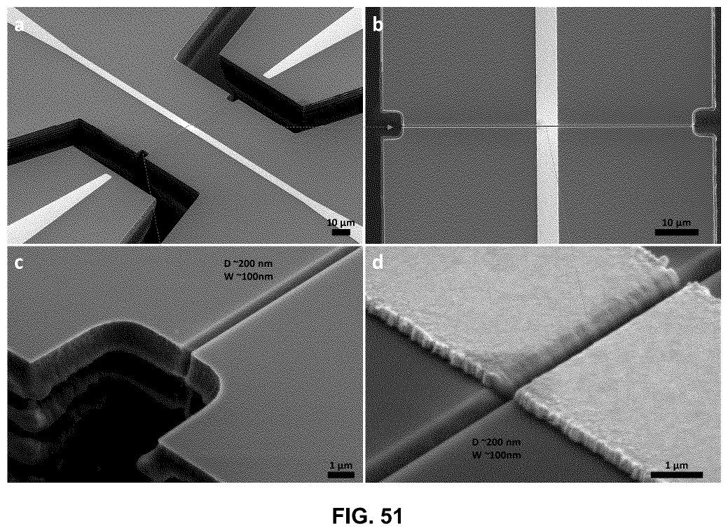

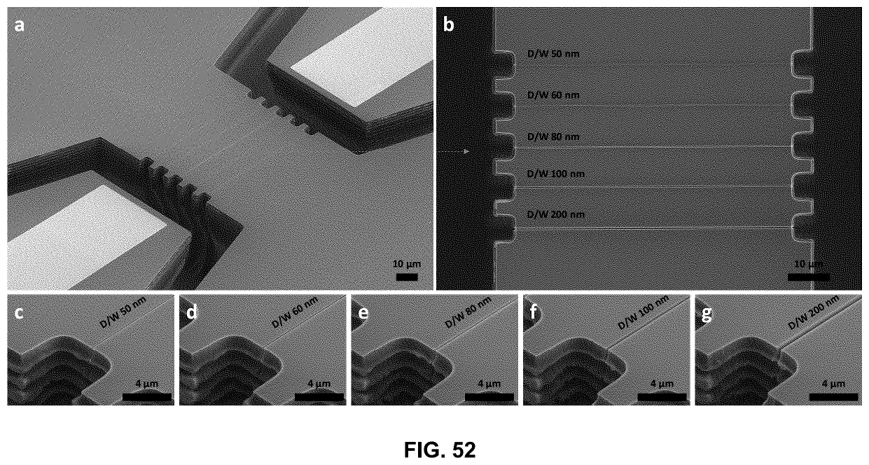

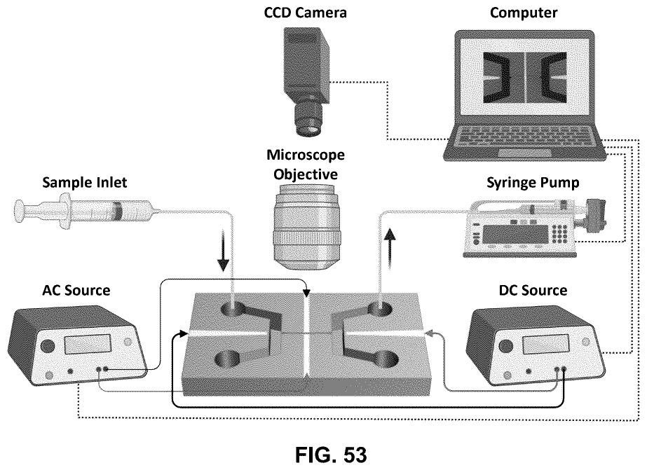

FIG. 50 shows the fabrication process flow of the example nanochannel assembly device. (a) Device fabrication begins with a 500 m thick, single-side polished, p-type silicon wafer with resistivity range of 0-100Ω cm. (b) The 4-inch Si wafer is patterned using photoresist (AZ 1512, Merck) and then (c) adhesion layer of 10 nm Cr and electrode metal of 100 nm Au were electron-beam evaporated (BJD-1800 E-Beam Evaporator 2, Temescal) and then (d) lifted off. (e) Photolithography was performed again, but with a different photoresist (AZ 12XT-20PL-5, Merck) to be used as a dry etch mask for Si dry etch process for the microchannels. (f) DRIE (Plasmalab 100, Oxford Instruments) was used to perform anisotropic 2-step etch process (Bosch process, or combination of isotropic plasma etch with SF6, and passivation with C4F8) to form 20 μm-wide, 40-60 μm-deep, microchannels. (g) After removing the PR dry etch mask, (h) the nanochannel was formed in between the microchannel regions by focused ion beam (FIB) etching (Scios DualBeam FIB/SEM, FEI). FIB etching was set at accelerating voltage of 30 kV and the currents were optimized between 10 pA and 30 pA to achieve nanochannels with width and depth of 50 nm/60 nm/80 nm/100 nm/200 nm. FIG. 51 shows the resultant nanochannel device. FIG. 52 shows a version of the device with 5 nanochannels, each with a varying width and depth, and without the centralized electrodes. FIG. 53 shows a laboratory schematic for utilizing and monitoring the device.

### Example 2. Nanochannel Assembly Methods

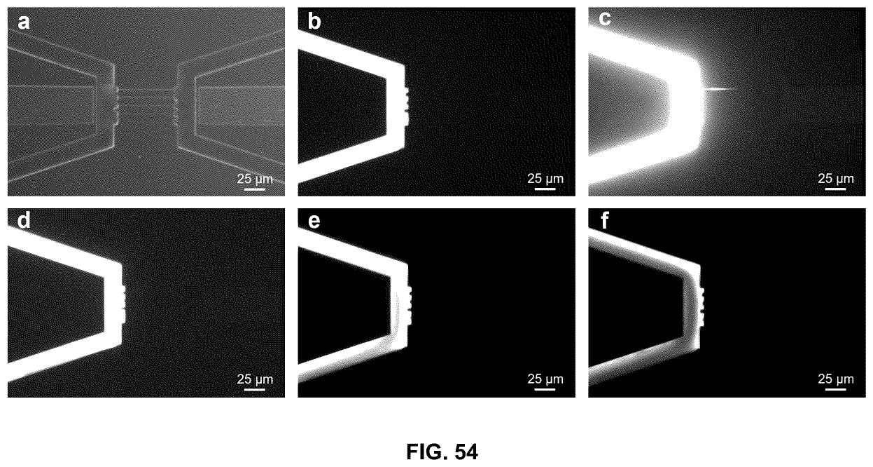

FIG. 54 shows loading of DNA into the channels of an example nanochannel assembly device with and without application of electrical signal. Panels a through c highlight channel wettability. Prior to use, the device was washed three times with isopropyl alcohol and treated with UV/ozone for thirty minutes. The brightfield image in panel a depicts the surface of the device after cleaning. Five nanochannels (length of 75 um) with different cross-sectional dimensions (from top to bottom: width and depth of 200 nm, 100 nm, 80 nm, 60 nm, and 50 nm) bridge two microchannels at the center of the field-of-view. Two electrodes flank the microchannels but do not directly contact the channel walls. The fluorescence image in panel b shows loading of one microchannel with fluorescent DNA. A sample containing 45 mM Tris-borate, 1 mM EDTA, 0.05% Tween 20, and 1 M fluorophore-labeled DNA was introduced to the reservoir for the microchannel at left. The fluorescence image in panel c shows wetting of the top nanochannel. A nonfluorescent buffer containing 45 mM Tris-borate, 1 mM EDTA, and 0.05% Tween 20 was introduced to the reservoir for the microchannel at right, and fluorescent DNA from the microchannel at left entered the top nanochannel upon wetting. Panels d through f show electric field-induced motion of DNA within a microchannel. The fluorescence image in panel d depicts sample in the microchannel at left and buffer in the microchannel at right prior to application of voltage between the two electrodes. Panel e shows repulsion of negatively charged fluorescent DNA away from the electrode at left in response to an applied voltage (ΔV=Vright electrode−Vleft electrode=2 V DC). Panel f shows DNA continuing to accumulate at the nanochannel entrances over time under applied voltage, demonstrating the feasibility of applying electric fields to induce DNA motion within the device.

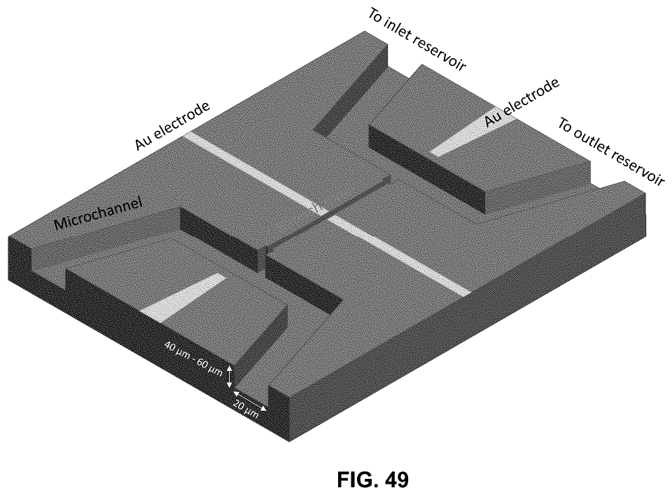

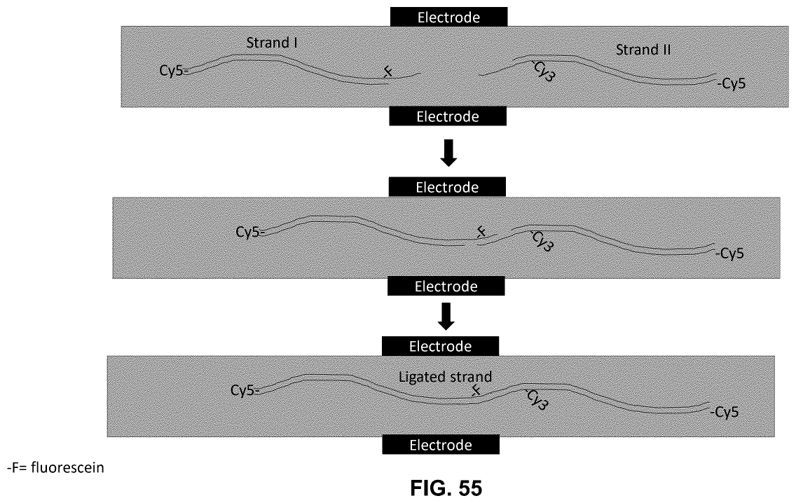

FIG. 55 illustrates the joining of two strands using the nanochannel chip design depicted in FIG. 49. A solution of 50 mM Tris-HCl, 1 mM ATP, 10 mM DTT, and 40 U/μL of T4 DNA ligase (New England Biolabs) containing a DNA sequence Strand I is added to the microchannel fluidic reservoir on the left-hand side of the chip. A solution of 50 mM Tris-HCl, 1 mM ATP, 10 mM DTT, and 40 U/μL of T4 DNA ligase containing a DNA sequence Strand II is added to the microchannel fluidic reservoir on the right-hand side of the chip. Strand I and Strand II each are labelled with a Cy5 fluorophore at one termini, while Strand I is labelled with fluorescein at the end distal to the Cy5, while Strand II is labelled with a Cy3 fluorophore at the end distal to the Cy5. The two strands bear complementary sticky ends as shown. One copy of each of the two strands are introduced into the nanochannel of a device under control of electrodes which are positioned centrally in the nanochannel (as shown) as well as at the distal ends of the nanochannel (not shown). The termini of each strand are positioned proximally to one another within the channel, again under electrical control. The strands are incubated and allowed to anneal, and MgCl2 is added to both reservoirs outside of the channel to an approximate final concentration of 10 mM. The Mg2+ diffuses through the channel so that the two strands can be joined by the activity of the T4-ligase, which is incubated at room temperature. The result is a single large duplex of ligated Strand I and Strand II. After the strands have been joined, the joined strand is moved from the channel under electric control.

What is claimed is:

1. A DNA assembly device comprising:
a chip having at least one nanochannel integrated therein, the at least one nanochannel having a reaction zone where ends of at least two distinct DNA molecules can be maintained proximate to one another; and
circuits and electrodes for controlling movement of the at least two distinct DNA molecules.

2. The device of claim 1, wherein movement of the at least two distinct DNA molecules is controlled electrically.

3. The device of claim 1, wherein the DNA molecules have lengths of 50-1,000 bases, or 1,000-10,000 bases, or 10,000-100,000 bases, or 100,000-1,000,000 bases, or 1,000,000-10,000,000 bases, or more than 10,000,000 bases.

4. The device of claim 1, wherein the chip contains multiple nanochannels, each of which can operate independently.

5. The device of claim 1, wherein the chip is a CMOS chip.

6. The device of claim 1, wherein the top of the at least one nanochannel is sealed.

7. The device of claim 1, wherein the surfaces of the at least one nanochannel are functionalized.

8. The device of claim 1, wherein at least one portion of the at least one nanochannel is constricted.

9. The device of claim 1, wherein the reaction zone is fluidically connected to an end of the at least one nanochannel.

10. The device of claim 1, wherein the reaction zone is configured along the length of the at least one nanochannel.

11. A system comprising:
first and second devices according to claim 1, wherein each of the respective nanochannels are fluidically connected.

12. A system comprising:
a device according to claim 4, wherein the device contains a network of nanochannels.

13. The system of claim 12, wherein the number of networked nanochannels is 1-10, or 10-100, or 100-1000, or 1000-10,000, or 10,000-100,000, or 100,000-1,000,000, or more than 1,000,000.

14. The system of claim 12, wherein the chip is a CMOS chip.

15. A method of joining two DNA molecules, comprising:
(a) introducing a first DNA molecule to a first nanochannel, wherein the first nanochannel contains a reaction zone;
(b) introducing a second DNA molecule strand to a second nanochannel, wherein the second nanochannel is also connected to the reaction zone; and
(c) electrically controlling the movement of the first and second DNA molecules strands to the reaction zone where the first and second nucleotide strands undergo a series of joining reactions.

16. The method of claim 15, wherein the first and second nanochannels are the same.

17. The method of claim 15, wherein each of the DNA molecules contain a mechanical blocking group.

18. The method of claim 15, further comprising removing the mechanical blocking groups following the joining of the first and second DNA molecules.

19. The method of claim 15, wherein the DNA molecules contain barcode elements for tracking.

20. The method of claim 15, wherein the DNA molecules have lengths of 50-1,000 bases, or 1,000-10,000 bases, or 10,000-100,000 bases, or 100,000-1,000,000 bases, or 1,000,000-10,000,000 bases, or more than 10,000,000 bases.
